(19) United States
(12) Patent Application Publication
(10) Pub. No.: US 2006/0075519 A1
(43) Pub. Date: Apr. 6, 2006

(54) Polypeptides having cellobiase activity and ploynucleotides encoding same

(75) Inventors: Martin Schulein, Copenhagen (DK); Torben Henriksen, Copenhagen (DE); Jan Lehmbeck, Vekso (DE)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 10/478,151

(22) PCT Filed: May 17, 2002
(86) PCT No.: PCT/DK02/00325

§ 371 Date: Oct. 20, 2005

(30) Foreign Application Priority Data
May 18, 2001 (DK) PA 2001 00798

(51) Int. Cl. A01H 1/00; C07H 21/04; C12P 21/06; C12N 9/42; C12N 5/04; C12N 15/82

(52) U.S. Cl. 800/284; 435/69.1; 435/209; 435/419; 435/468; 536/23.2

(57) ABSTRACT

The present invention relates to polypeptides having cellobiase activity and polynucleotides having a nucleotide sequence which encodes for the polypeptides. The invention also relates to nucleic acid constructs, vectors, and host cells comprising the nucleic acid constructs as well as methods for producing and using the polypeptides.

### FIELD OF THE INVENTION

The present invention relates to polypeptides having cellobiase (also referred to as beta-glucosidase) activity and polynucleotides having a nucleotide sequence which encodes for the polypeptides. The invention also relates to nucleic acid constructs, vectors, and host cells comprising the nucleic acid constructs as well as methods for producing and using the polypeptides.

### BACKGROUND OF THE INVENTION

Cellobiases are important enzymes for degradation of biomass. This use of cellobiases is a key process in the production of ethanol from plant material. For environmental reasons ethanol is an attractive fuel alternative compared to petroleum based fuels. Other uses of cellobiases include application in the fruit juice industry for reduction of bitter compounds like quercetin.

It is an object of the present invention to provide polypeptides having cellobiase (also referred to as beta-glucosidase) activity and polynucleotides encoding the polypeptides.

### SUMMARY OF THE INVENTION

In a first aspect the present invention relates to a polypeptide having cellobiase activity, selected from the group consisting of:
(a) a polypeptide comprising an amino acid sequence which has at least 80% identity with amino acids 1 to 842 of SEQ ID NO:2 or which has at least 90% identity with amino acids 1 to 351 of SEQ ID NO:2;
(b) a polypeptide comprising an amino acid sequence which has at least 80% identity with the polypeptide encoded by the cellobiase encoding part of the nucleotide sequence inserted into a plasmid present in E. coli DSM 14240;
(c) a polypeptide which is encoded by a nucleotide sequence which hybridizes under medium stringency conditions with a polynucleotide probe selected from the group consisting of
   (i) the complementary strand of nucleotides 87 to 2612 of SEQ ID NO:1, and
   (ii) the complementary strand of nucleotides 87 to 1139 of SEQ ID NO:1;
(d) a fragment of (a), (b) or (c) that has cellobiase activity.

In a second aspect the present invention relates to polynucleotides having a nucleotide sequence which encodes for the polypeptide of the invention.

In a third aspect the present invention relates to a nucleic acid construct comprising the nucleotide sequence, which encodes for the polypeptide of the invention, operably linked to one or more control sequences that direct the production of the polypeptide in a suitable host.

In a fourth aspect the present invention relates to a recombinant expression vector comprising the nucleic acid construct of the invention.

In a fifth aspect the present invention relates to a recombinant host cell comprising the nucleic acid construct of the invention.

In a sixth aspect the present invention relates to a method for producing a polypeptide of the invention, the method comprising:
(a) cultivating a strain, which in its wild-type form is capable of producing the polypeptide, to produce the polypeptide; and
(b) recovering the polypeptide.

In a seventh aspect the present invention relates to a method for producing a polypeptide of the invention, the method comprising:
(a) cultivating a recombinant host cell of the invention under conditions conducive for production of the polypeptide; and
(b) recovering the polypeptide.

Other aspects of the present invention will be apparent from the below description and from the appended claims.

Definitions

Prior to discussing the present invention in further details, the following terms and conventions will first be defined:

Substantially pure polypeptide: In the present context, the term “substantially pure polypeptide” means a polypeptide preparation which contains at the most 10% by weight of other polypeptide material with which it is natively associated (lower percentages of other polypeptide material are preferred, e.g. at the most 8% by weight, at the most 6% by weight, at the most 5% by weight, at the most 4% at the most 3% by weight, at the most 2% by weight, at the most 1% by weight, and at the most ½% by weight). Thus, it is preferred that the substantially pure polypeptide is at least 92% pure, i.e. that the polypeptide constitutes at least 92% by weight of the total polypeptide material present in the preparation, and higher percentages are preferred such as at least 94% pure, at least 95% pure, at least 96% pure, at least 96% pure, at least 97% pure, at least 98% pure, at least 99%, and at the most 99.5% pure. The polypeptides disclosed herein are preferably in a substantially pure form. In particular, it is preferred that the polypeptides disclosed herein are in “essentially pure form”, i.e. that the polypeptide preparation is essentially free of other polypeptide material with which it is natively associated. This can be accomplished, for example, by preparing the polypeptide by means of well-known recombinant methods. Herein, the term “substantially pure polypeptide” is synonymous with the terms “isolated polypeptide” and “polypeptide in isolated form”. Cellobiase activity: The term “cellobiase activity” is defined herein as a beta-glucosidase activity, as defined in the enzyme class EC 3.2.1.21, which catalyzes the hydrolysis of terminal non-reducing beta-D-glucose residues with release of beta-D-glucose. For purposes of the present invention, cellobiase activity is determined according to the procedure described in “Cellobiase assay” in the Examples section. One unit of cellobiase activity (CBU) is defined as 2 μmole of glucose produced per minute at 40° C., pH 5.

The polypeptides of the present invention should preferably have at least 20% of the cellobiase activity of the polypeptide consisting of the amino acid sequence shown as amino acids 1 to 842 of SEQ ID NO:2. In a particular preferred embodiment, the polypeptides should have at least 40%, such as at least 50%, preferably at least 60%, such as at least 70%, more preferably at least 80%, such as at least 90%, most preferably at least 95%, such as about or at least 100% of the cellobiase activity of the polypeptide consisting of the amino acid sequence shown as amino acids 1 to 842 of SEQ ID NO:2.

Identity: In the present context, the homology between two amino acid sequences or between two nucleotide sequences is described by the parameter “identity”.

For purposes of the present invention, the degree of identity between two amino acid sequences is determined by a Needleman-Wunsch alignment, useful for both protein and DNA alignments. For protein alignments the default scoring matrix used is BLOSUM50, and the penalty for the first residue in a gap is −12, while the penalty for additional residues in a gap is −2. The alignment may be made with the Align software from the FASTA package version v20u6 (W. R. Pearson and D. J. Lipman (1988), “Improved Tools for Biological Sequence Analysis”, PNAS 85:2444-2448; and W. R. Pearson (1990) “Rapid and Sensitive Sequence Comparison with FASTP and FASTA”, Methods in Enzymology, 183:63-98).

The degree of identity between two nucleotide sequences may be determined using the same algorithm and software package as described above using the identity matrix as the default scoring matrix. The penalty for the first residue in a gap is −16, while the penalty for additional residues in a gap is −4.

Fragment: When used herein, a “fragment” of SEQ ID NO:2 is a polypeptide having one or more amino acids deleted from the amino and/or carboxyl terminus of this amino acid sequence. Preferably, a fragment contains at least 351 amino acid residues, e.g., amino acids 1 to 351 of SEQ ID NO:2.

Allelic variant: In the present context, the term “allelic variant” denotes any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Substantially pure polynucleotide: The term “substantially pure polynucleotide” as used herein refers to a polynucleotide preparation, wherein the polynucleotide has been removed from its natural genetic milieu, and is thus free of other extraneous or unwanted coding sequences and is in a form suitable for use within genetically engineered protein production systems. Thus, a substantially pure polynucleotide contains at the most 10% by weight of other polynucleotide material with which it is natively associated (lower percentages of other polynucleotide material are preferred, e.g. at the most 8% by weight, at the most 6% by weight, at the most 5% by weight, at the most 4% at the most 3% by weight, at the most 2% by weight, at the most 1% by weight, and at the most ½% by weight). A substantially pure polynucleotide may, however, include naturally occurring 5′ and 3′ untranslated regions, such as promoters and terminators. It is preferred that the substantially pure polynucleotide is at least 92% pure, i.e. that the polynucleotide constitutes at least 92% by weight of the total polynucleotide material present in the preparation, and higher percentages are preferred such as at least 94% pure, at least 95% pure, at least 96% pure, at least 96% pure, at least 97% pure, at least 98% pure, at least 99%, and at the most 99.5% pure. The polynucleotides disclosed herein are preferably in a substantially pure form. In particular, it is preferred that the polynucleotides disclosed herein are in “essentially pure form”, i.e. that the polynucleotide preparation is essentially free of other polynucleotide material with which it is natively associated. Herein, the term “substantially pure polynucleotide” is synonymous with the terms “isolated polynucleotide” and “polynucleotide in isolated form”.

Modification(s): In the context of the present invention the term “modification(s)” is intended to mean any chemical modification of the polypeptide consisting of the amino acid sequence shown as amino acids 1 to 842 of SEQ ID NO:2 as well as genetic manipulation of the DNA encoding that polypeptide. The modification(s) can be replacement(s) of the amino acid side chain(s), substitution(s), deletion(s) and/or insertions(s) in or at the amino acid(s) of interest.

Artificial variant: When used herein, the term “artificial variant” means a polypeptide having cellobiase activity, which has been produced by an organism which is expressing a modified gene as compared to SEQ ID NO:1. The modified gene, from which said variant is produced when expressed in a suitable host, is obtained through human intervention by modification of the nucleotide sequence disclosed in SEQ ID NO:1.

cDNA: The term “cDNA” when used in the present context, is intended to cover a DNA molecule which can be prepared by reverse transcription from a mature, spliced, mRNA molecule derived from a eukaryotic cell. cDNA lacks the intron sequences that are usually present in the corresponding genomic DNA. The initial, primary RNA transcript is a precursor to mRNA and it goes through a series of processing events before appearing as mature spliced mRNA. These events include the removal of intron sequences by a process called splicing. When cDNA is derived from mRNA it therefore lacks intron sequences.

Nucleic acid construct: When used herein, the term “nucleic acid construct” means a nucleic acid molecule, either single- or double-stranded, which is isolated from a naturally occurring gene or which has been modified to contain segments of nucleic acids in a manner that would not otherwise exist in nature. The term nucleic acid construct is synonymous with the term “expression cassette” when the nucleic acid construct contains the control sequences required for expression of a coding sequence of the present invention.

Control sequence: The term “control sequences” is defined herein to include all components, which are necessary or advantageous for the expression of a polypeptide of the present invention. Each control sequence may be native or foreign to the nucleotide sequence encoding the polypeptide. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleotide sequence encoding a polypeptide.

Operably linked: The term “operably linked” is defined herein as a configuration in which a control sequence is appropriately placed at a position relative to the coding sequence of the DNA sequence such that the control sequence directs the expression of a polypeptide.

Coding sequence: When used herein the term “coding sequence” is intended to cover a nucleotide sequence, which directly specifies the amino acid sequence of its protein product. The boundaries of the coding sequence are generally determined by an open reading frame, which usually begins with the ATG start codon. The coding sequence typically include DNA, cDNA, and recombinant nucleotide sequences.

Expression: In the present context, the term “expression” includes any step involved in the production of the polypeptide including, but not limited to, transcription, post-transcriptional modification, translation, post-translational modification, and secretion.

Expression vector: In the present context, the term “expression vector” covers a DNA molecule, linear or circular, that comprises a segment encoding a polypeptide of the invention, and which is operably linked to additional segments that provide for its transcription.

Host cell: The term “host cell”, as used herein, includes any cell type which is susceptible to transformation with a nucleic acid construct.

The terms “polynucleotide probe”, “hybridization” as well as the various stringency conditions are defined in the section entitled “Polypeptides Having Cellobiase Activity”.

### DETAILED DESCRIPTION OF THE INVENTION

Polypeptides Having Cellobiase Activity

In a first embodiment, the present invention relates to polypeptides having cellobiase activity and where the polypeptides comprises, preferably consists of, an amino acid sequence which has a degree of identity to amino acids 1 to 842 of SEQ ID NO:2 (i.e., the mature polypeptide) of at least 65%, preferably at least 70%, e.g. at least 75%, more preferably at least 80%, such as at least 85%, even more preferably at least 90%, most preferably at least 95%, e.g. at least 96%, such as at least 97%, and even most preferably at least 98%, such as at least 99% (hereinafter “homologous polypeptides”). In an interesting embodiment, the amino acid sequence differs by at the most ten amino acids (e.g. by ten amino acids), in particular by at the most five amino acids (e.g. by five amino acids), such as by at the most four amino acids (e.g. by four amino acids), e.g. by at the most three amino acids (e.g. by three amino acids) from amino acids 1 to 842 of SEQ ID NO:2. In a particular interesting embodiment, the amino acid sequence differs by at the most two amino acids (e.g. by two amino acids), such as by one amino acid from amino acids 1 to 842 of SEQ ID NO:2.

In another embodiment the polypeptides comprises, preferably consists of, an amino acid sequence which has a degree of identity to amino acids 1 to 351 of SEQ ID NO:2 (i.e., the catalytic core) of at least 65%, preferably at least 70%, e.g. at least 75%, more preferably at least 80%, such as at least 85%, even more preferably at least 90%, most preferably at least 95%, e.g. at least 96%, such as at least 97%, and even most preferably at least 98%, such as at least 99%.

Aligning the polypeptide consisting of the amino acid sequence shown as amino acids 1 to 842 of SEQ ID NO:2 with the closest prior art and using the method described above (see the section entitled “Definitions”), the following identity percentages were obtained:
Aspergillus aculeatus: 79.6%,
Aspergillus niger: 76.9%,
Aspergillus kawasachi: 77.0%.

Aligning the polypeptide consisting of the amino acid sequence shown as amino acids 1 to 351 of SEQ ID NO:2 (i.e. the catalytic core) with the closest prior art, the following identity percentages were obtained:
Aspergillus aculeatus: 86.6%,
Aspergillus niger: 81.9%,
Aspergillus kawasachi: 80.3%,
Aspergillus nidulans: 25.1%.

Preferably, the polypeptides of the present invention comprise the amino acid sequence of SEQ ID NO:2; an allelic variant thereof; or a fragment thereof that has cellobiase activity. In another preferred embodiment, the polypeptide of the present invention comprises amino acids 1 to 842 of SEQ ID NO:2. In a further preferred embodiment, the polypeptide consists of amino acids 1 to 842 of SEQ ID NO:2.

The polypeptide of the invention may be a wild-type cellobiase identified and isolated from a natural source. Such wild-type polypeptides may be specifically screened for by standard techniques known in the art. Furthermore, the polypeptide of the invention may be prepared by the DNA shuffling technique, such as described in J. E. Ness et al. Nature Biotechnology 17, 893-896 (1999). Moreover, the polypeptide of the invention may be an artificial variant which comprises, preferably consists of, an amino acid sequence that has at least one substitution, deletion and/or insertion of an amino acid as compared to amino acids 1 to 842 of SEQ ID NO:2. Such artificial variants may be constructed by standard techniques known in the art, such as by site-directed/random mutagenesis of the polypeptide comprising the amino acid sequence shown as amino acids 1 to 842 of SEQ ID NO:2. In one embodiment of the invention, amino acid changes (in the artificial variant as well as in wild-type polypeptides) are of a minor nature, that is conservative amino acid substitutions that do not significantly affect the folding and/or activity of the protein; small deletions, typically of one to about 30 amino acids; small amino- or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine, valine and methionine), aromatic amino acids (phenylalanine, tryptophan and tyrosine), and small amino acids (glycine, alanine, serine and threonine). Amino acid substitutions which do not generally alter the specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly as well as these in reverse.

In an interesting embodiment of the invention, the amino acid changes are of such a nature that the physico-chemical properties of the polypeptides are altered. For example, amino acid changes may be performed, which improve the thermal stability of the polypeptide, which alter the substrate specificity, which changes the pH optimum, and the like.

Preferably, the number of such substitutions, deletions and/or insertions as compared to amino acids 1 to 842 of SEQ ID NO:2 is at the most 10, such as at the most 9, e.g. at the most 8, more preferably at the most 7, e.g. at the most 6, such as at the most 5, most preferably at the most 4, e.g. at the most 3, such as at the most 2, in particular at the most 1.

The present inventors have isolated a gene encoding a polypeptide having cellobiase activity from Aspergillus oryzae and inserted it into plasmid pJaL621 (see Example 1) which was inserted in E. coli. The E. coli strain harboring the gene was deposited according to the Budapest Treaty on the International Recognition of the Deposits of Microorganisms for the Purpose of Patent Procedures on 2001 Apr. 19 at the DSMZ-Deutsche Sammlung von Mikroorganismen und Zellkulturen GmbH, Mascheroder Weg 1B, D-38124 Braunschweig, Germany, and designated the accession No. DSM 14240.

Thus, in a second embodiment, the present invention relates to polypeptides comprising, preferably consisting of, an amino acid sequence which has at least 65% identity with the polypeptide encoded by the cellobiase encoding part of the nucleotide sequence inserted into a plasmid present in E. coli DSM 14240. In an interesting embodiment of the invention, the polypeptide comprises, preferably consists of, an amino acid sequence which has at least 70%, e.g. at least 75%, preferably at least 80%, such as at least 85%, more preferably at least 90%, most preferably at least 95%, e.g. at least 96%, such as at least 97%, and even most preferably at least 98%, such as at least 99% identity with the polypeptide encoded by the cellobiase encoding part of the nucleotide sequence inserted into a plasmid present in E. coli DSM 14240 (hereinafter “homologous polypeptides”). In an interesting embodiment, the amino acid sequence differs by at the most ten amino acids (e.g. by ten amino acids), in particular by at the most five amino acids (e.g. by five amino acids), such as by at the most four amino acids (e.g. by four amino acids), e.g. by at the most three amino acids (e.g. by three amino acids) from the polypeptide encoded by the cellobiase encoding part of the nucleotide sequence inserted into a plasmid present in E. coli DSM 14240. In a particular interesting embodiment, the amino acid sequence differs by at the most two amino acids (e.g. by two amino acids), such as by one amino acid from the polypeptide encoded by the cellobiase encoding part of the nucleotide sequence inserted into a plasmid present in E. coli DSM 14240.

Preferably, the polypeptides of the present invention comprise the amino acid sequence of the polypeptide encoded by the cellobiase encoding part of the nucleotide sequence inserted into a plasmid present in E. coli DSM 14240. In another preferred embodiment, the polypeptide of the present invention consists of the amino acid sequence of the polypeptide encoded by the cellobiase encoding part of the nucleotide sequence inserted into a plasmid present in E. coli DSM 14240.

In a similar way as described above, the polypeptide of the invention may be an artificial variant which comprises, preferably consists of, an amino acid sequence that has at least one substitution, deletion and/or insertion of an amino acid as compared to the amino acid sequence encoded by the cellobiase encoding part of the nucleotide sequence inserted into a plasmid present in E. coli DSM 14240.

In a third embodiment, the present invention relates to polypeptides having cellobiase activity which are encoded by nucleotide sequences which hybridize under very low stringency conditions, preferably under low stringency conditions, more preferably under medium stringency conditions, more preferably under medium-high stringency conditions, even more preferably under high stringency conditions, and most preferably under very high stringency conditions with a polynucleotide probe selected from the group consisting of (i) the complementary strand of nucleotides 87 to 2612 of SEQ ID NO:1, and (ii) the complementary strand of nucleotides 87 to 1139 of SEQ ID NO:1 (J. Sambrook, E. F. Fritsch, and T. Maniatus, 1989, Molecular Cloning, A Laboratory Manual, 2d edition, Cold Spring Harbor, N.Y.).

The nucleotide sequence of SEQ ID NO:1 or a subsequence thereof, as well as the amino acid sequence of SEQ ID NO:2 or a fragment thereof, may be used to design a polynucleotide probe to identify and clone DNA encoding polypeptides having cellobiase activity from strains of different genera or species according to methods well known in the art. In particular, such probes can be used for hybridization with the genomic or cDNA of the genus or species of interest, following standard Southern blotting procedures, in order to identify and isolate the corresponding gene therein. Such probes can be considerably shorter than the entire sequence, but should be at least 15, preferably at least 25, more preferably at least 35 nucleotides in length, such as at least 70 nucleotides in length. It is, however, preferred that the polynucleotide probe is at least 100 nucleotides in length. For example, the polynucleotide probe may be at least 200 nucleotides in length, at least 300 nucleotides in length, at least 400 nucleotides in length or at least 500 nucleotides in length. Even longer probes may be used, e.g., polynucleotide probes which are at least 600 nucleotides in length, at least 700 nucleotides in length, at least 800 nucleotides in length, or at least 900 nucleotides in length. Both DNA and RNA probes can be used. The probes are typically labeled for detecting the corresponding gene (for example, with 32P, 3H, 35S, biotin, or avidin).

Thus, a genomic DNA or cDNA library prepared from such other organisms may be screened for DNA which hybridizes with the probes described above and which encodes a polypeptide having cellobiase activity. Genomic or other DNA from such other organisms may be separated by agarose or polyacrylamide gel electrophoresis, or other separation techniques. DNA from the libraries or the separated DNA may be transferred to, and immobilized, on nitrocellulose or other suitable carrier materials. In order to identify a clone or DNA which is homologous with SEQ ID NO:1 the carrier material with the immobilized DNA is used in a Southern blot.

For purposes of the present invention, hybridization indicates that the nucleotide sequence hybridizes to a labeled polynucleotide probe which hybridizes to the nucleotide sequence shown in SEQ ID NO:1 under very low to very high stringency conditions. Molecules to which the polynucleotide probe hybridizes under these conditions may be detected using X-ray film or by any other method known in the art. Whenever the term “polynucleotide probe” is used in the present context, it is to be understood that such a probe contains at least 15 nucleotides.

In an interesting embodiment, the polynucleotide probe is the complementary strand of nucleotides 87 to 1139 of SEQ ID NO:1.

In another interesting embodiment, the polynucleotide probe is the complementary strand of the nucleotide sequence which encodes the polypeptide of SEQ ID NO:2. In a further interesting embodiment, the polynucleotide probe is the complementary strand of SEQ ID NO:1. In a still further interesting embodiment, the polynucleotide probe is the complementary strand of the mature polypeptide coding region of SEQ ID NO:1. In another interesting embodiment, the polynucleotide probe is the complementary strand of the nucleotide sequence contained in plasmid pJaL621 which is contained in E. coli DSM 14240. In still another interesting embodiment, the polynucleotide probe is the complementary strand of the mature polypeptide coding region contained in plasmid pJaL621 which is contained in E. coli DSM 14240.

For long probes of at least 100 nucleotides in length, very low to very high stringency conditions are defined as prehybridization and hybridization at 42° C. in 5×SSPE, 1.0% SDS, 5× Denhardt's solution, 100 μg/ml sheared and denatured salmon sperm DNA, following standard Southern blotting procedures. Preferably, the long probes of at least 100 nucleotides do not contain more than 1000 nucleotides. For long probes of at least 100 nucleotides in length, the carrier material is finally washed three times each for 15 minutes using 2×SSC, 0.1% SDS at 42° C. (very low stringency), preferably washed three times each for 15 minutes using 0.5×SSC, 0.1% SDS at 42° C. (low stringency), more preferably washed three times each for 15 minutes using 0.2×SSC, 0.1% SDS at 42° C. (medium stringency), even more preferably washed three times each for 15 minutes using 0.2×SSC, 0.1% SDS at 55° C. (medium-high stringency), most preferably washed three times each for 15 minutes using 0.1×SSC, 0.1% SDS at 60° C. (high stringency), in particular washed three times each for 15 minutes using 0.1×SSC, 0.1% SDS at 68° C. (very high stringency).

Although not particularly preferred, it is contemplated that shorter probes, e.g. probes which are from about 15 to 99 nucleotides in length, such as from about 15 to about 70 nucleotides in length, may be also be used. For such short probes, stringency conditions are defined as prehybridization, hybridization, and washing post-hybridization at 5° C. to 10° C. below the calculated Tm using the calculation according to Bolton and McCarthy (1962, Proceedings of the National Academy of Sciences USA 48:1390) in 0.9 M NaCl, 0.09 M Tris-HCl pH 7.6, 6 mM EDTA, 0.5% NP40, 1× Denhardt's solution, 1 mM sodium pyrophosphate, 1 mM sodium monobasic phosphate, 0.1 mM ATP, and 0.2 mg of yeast RNA per ml following standard Southern blotting procedures.

For short probes which are about 15 nucleotides to 99 nucleotides in length, the carrier material is washed once in 6×SCC plus 0.1% SDS for 15 minutes and twice each for 15 minutes using 6×SSC at 5° C. to 10° C. below the calculated Tm.

Sources for Polypeptides Having Cellobiase Activity

A polypeptide of the present invention may be obtained from microorganisms of any genus. For purposes of the present invention, the term “obtained from” as used herein shall mean that the polypeptide encoded by the nucleotide sequence is produced by a cell in which the nucleotide sequence is naturally present or into which the nucleotide sequence has been inserted. In a preferred embodiment, the polypeptide is secreted extracellularly.

A polypeptide of the present invention may be a bacterial polypeptide. For example, the polypeptide may be a gram positive bacterial polypeptide such as a Bacillus polypeptide, e.g., a Bacillus alkalophilus, Bacillus amyloliquefaciens, Bacillus brevis, Bacillus circulans, Bacillus coagulans, Bacillus lautus, Bacillus lentus, Bacillus licheniformis, Bacillus megaterium, Bacillus stearothermophilus, Bacillus subtilis, or Bacillus thuringiensis polypeptide; or a Streptomyces polypeptide, e.g., a Streptomyces lividans or Streptomyces murinus polypeptide; or a gram negative bacterial polypeptide, e.g., an E. coli or a Pseudomonas sp. polypeptide.

A polypeptide of the present invention may be a fungal polypeptide, and more preferably a yeast polypeptide such as a Candida, Kluyveromyces, Pichia, Saccharomyces, Schizosaccharomyces, or Yarrowia polypeptide; or more preferably a filamentous fungal polypeptide such as an Acremonium, Aspergillus, Aureobasidium, Cryptococcus, Filibasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, or Trichoderma polypeptide.

In an interesting embodiment, the polypeptide is a Saccharomyces carlsbergensis, Saccharomyces cerevisiae, Saccharomyces diastaticus, Saccharomyces douglasii, Saccharomyces kluyveri, Saccharomyces norbensis or Saccharomyces oviformis polypeptide.

In another interesting embodiment, the polypeptide is an Aspergillus aculeatus, Aspergillus awamori, Aspergillus foetidus, Aspergillus japonicus, Aspergillus nidulans, Aspergillus niger, Aspergillus oryzae, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Trichoderma harzianum, Trichoderma koningii, Trichoderma longibrachiatum, Trichoderma reesei, or Trichoderma viride polypeptide.

In a preferred embodiment, the polypeptide is an Aspergillus oryzae polypeptide, and most preferably an E. coli DSM 14240 polypeptide, e.g., the polypeptide consisting of the amino acid sequence 1 to 842 of SEQ ID NO:2.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen und Zellkulturen GmbH (DSM), Centraalbureau Voor Schimmelcultures (CBS), and Agricultural Research Service Patent Culture Collection, Northern Regional Research Center (NRRL).

Furthermore, such polypeptides may be identified and obtained from other sources including microorganisms isolated from nature (e.g., soil, composts, water, etc.) using the above-mentioned probes. Techniques for isolating microorganisms from natural habitats are well known in the art. The nucleotide sequence may then be derived by similarly screening a genomic or cDNA library of another microorganism. Once a nucleotide sequence encoding a polypeptide has been detected with the probe(s), the sequence may be isolated or cloned by utilizing techniques which are known to those of ordinary skill in the art (see, e.g., Sambrook et al., 1989, supra).

Polypeptides encoded by nucleotide sequences of the present invention also include fused polypeptides or cleavable fusion polypeptides in which another polypeptide is fused at the N-terminus or the C-terminus of the polypeptide or fragment thereof. A fused polypeptide is produced by fusing a nucleotide sequence (or a portion thereof) encoding another polypeptide to a nucleotide sequence (or a portion thereof) of the present invention. Techniques for producing fusion polypeptides are known in the art, and include ligating the coding sequences encoding the polypeptides so that they are in frame and that expression of the fused polypeptide is under control of the same promoter(s) and terminator.

Polynucleotides and Nucleotide Sequences

The present invention also relates to polynucleotides having a nucleotide sequence which encodes for a polypeptide of the invention. In particular, the present invention relates to polynucleotides consisting of a nucleotide sequence which encodes for a polypeptide of the invention. In a preferred embodiment, the nucleotide sequence is set forth in SEQ ID NO:1. In a more preferred embodiment, the nucleotide sequence is the mature polypeptide coding region of SEQ ID NO:1. In another more preferred embodiment, the nucleotide sequence is the mature polypeptide coding region contained in plasmid pJaL621 that is contained in E. coli DSM 14240. The present invention also encompasses polynucleotides having, preferably consisting of, nucleotide sequences which encode a polypeptide consisting of the amino acid sequence of SEQ ID NO:2 or the mature polypeptide thereof, which differ from SEQ ID NO:1 by virtue of the degeneracy of the genetic code.

The present invention also relates to polynucleotides having, preferably consisting of, a subsequence of SEQ ID NO:1 which encode fragments of SEQ ID NO:2 that have cellobiase activity. A subsequence of SEQ ID NO:1 is a nucleotide sequence encompassed by SEQ ID NO:1 except that one or more nucleotides from the 5′ and/or 3′ end have been deleted.

The present invention also relates to polynucleotides having, preferably consisting of, a modified nucleotide sequence which comprises at least one modification in the mature polypeptide coding sequence of SEQ ID NO:1, and where the modified nucleotide sequence encodes a polypeptide which consists of amino acids 1 to 842 of SEQ ID NO:2.

The techniques used to isolate or clone a nucleotide sequence encoding a polypeptide are known in the art and include isolation from genomic DNA, preparation from cDNA, or a combination thereof. The cloning of the nucleotide sequences of the present invention from such genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other amplification procedures such as ligase chain reaction (LCR), ligated activated transcription (LAT) and nucleotide sequence-based amplification (NASBA) may be used. The nucleotide sequence may be cloned from a strain of Aspergillus, or another or related organism and thus, for example, may be an allelic or species variant of the polypeptide encoding region of the nucleotide sequence.

The nucleotide sequence may be obtained by standard cloning procedures used in genetic engineering to relocate the nucleotide sequence from its natural location to a different site where it will be reproduced. The cloning procedures may involve excision and isolation of a desired fragment comprising the nucleotide sequence encoding the polypeptide, insertion of the fragment into a vector molecule, and incorporation of the recombinant vector into a host cell where multiple copies or clones of the nucleotide sequence will be replicated. The nucleotide sequence may be of genomic, cDNA, RNA, semisynthetic, synthetic origin, or any combinations thereof.

The present invention also relates to a polynucleotide having, preferably consisting of, a nucleotide sequence which has at least 65% identity with nucleotides 87 to 2612 of SEQ ID NO:1. Preferably, the nucleotide sequence has at least 70% identity, e.g. at least 80% identity, such as at least 90% identity, more preferably at least 95% identity, such as at least 96% identity, e.g. at least 97% identity, even more preferably at least 98% identity, such as at least 99% with nucleotides 87 to 2612 of SEQ ID NO:1. Preferably, the nucleotide sequence encodes a polypeptide having cellobiase activity. The degree of identity between two nucleotide sequences is determined as described previously (see the section entitled “Definitions”). Preferably, the nucleotide sequence comprises nucleotides 87 to 2612 of SEQ ID NO:1. In an even more preferred embodiment, the nucleotide sequence consists of nucleotides 87 to 2612 of SEQ ID NO:1.

In another interesting aspect, the present invention relates to a polynucleotide having, preferably consisting of, a nucleotide sequence which has at least 65% identity with the cellobiase encoding part of the nucleotide sequence inserted into a plasmid present in E. coli DSM 14240. In a preferred embodiment, the degree of identity with the cellobiase encoding part of the nucleotide sequence inserted into a plasmid present in E. coli DSM 14240 is at least 70%, e.g. at least 80%, such as at least 90%, more preferably at least 95%, such as at least 96%, e.g. at least 97%, even more preferably at least 98%, such as at least 99%. Preferably, the nucleotide sequence comprises the cellobiase encoding part of the nucleotide sequence inserted into a plasmid present in E. coli DSM 14240. In an even more preferred embodiment, the nucleotide sequence consists of the cellobiase encoding part of the nucleotide sequence inserted into a plasmid present in E. coli DSM 14240.

Modification of a nucleotide sequence encoding a polypeptide of the present invention may be necessary for the synthesis of a polypeptide, which comprises an amino acid sequence that has at least one substitution, deletion and/or insertion as compared to amino acids 1 to 842 of SEQ ID NO:2. These artificial variants may differ in some engineered way from the polypeptide isolated from its native source, e.g., variants that differ in specific activity, thermostability, pH optimum, or the like.

It will be apparent to those skilled in the art that such modifications can be made outside the regions critical to the function of the molecule and still result in an active polypeptide. Amino acid residues essential to the activity of the polypeptide encoded by the nucleotide sequence of the invention, and therefore preferably not subject to modification, such as substitution, may be identified according to procedures known in the art, such as site-directed mutagenesis or alanine-scanning mutagenesis (see, e.g., Cunningham and Wells, 1989, Science 244: 1081-1085). In the latter technique, mutations are introduced at every positively charged residue in the molecule, and the resultant mutant molecules are tested for cellobiase activity to identify amino acid residues that are critical to the activity of the molecule. Sites of substrate-enzyme interaction can also be determined by analysis of the three-dimensional structure as determined by such techniques as nuclear magnetic resonance analysis, crystallography or photoaffinity labelling (see, e.g., de Vos et al., 1992, Science 255: 306-312; Smith et al., 1992, Journal of Molecular Biology 224: 899-904; Wlodaver et al., 1992, FEBS Letters 309: 59-64).

Moreover, a nucleotide sequence encoding a polypeptide of the present invention may be modified by introduction of nucleotide substitutions which do not give rise to another amino acid sequence of the polypeptide encoded by the nucleotide sequence, but which correspond to the codon usage of the host organism intended for production of the enzyme.

The introduction of a mutation into the nucleotide sequence to exchange one nucleotide for another nucleotide may be accomplished by site-directed mutagenesis using any of the methods known in the art. Particularly useful is the procedure, which utilizes a supercoiled, double stranded DNA vector with an insert of interest and two synthetic primers containing the desired mutation. The oligonucleotide primers, each complementary to opposite strands of the vector, extend during temperature cycling by means of Pfu DNA polymerase. On incorporation of the primers, a mutated plasmid containing staggered nicks is generated. Following temperature cycling, the product is treated with DpnI which is specific for methylated and hemimethylated DNA to digest the parental DNA template and to select for mutation-containing synthesized DNA. Other procedures known in the art may also be used. For a general description of nucleotide substitution, see, e.g., Ford et al., 1991, Protein Expression and Purification 2: 95-107.

The present invention also relates to a polynucleotide having, preferably consisting of, a nucleotide sequence which encodes a polypeptide having cellobiase activity, and which hybridizes under very low stringency conditions, preferably under low stringency conditions, more preferably under medium stringency conditions, more preferably under medium-high stringency conditions, even more preferably under high stringency conditions, and most preferably under very high stringency conditions with a polynucleotide probe selected from the group consisting of (i) the complementary strand of nucleotides 87 to 2612 of SEQ ID NO:1, (ii) the complementary strand of nucleotides 87 to 1139 of SEQ ID NO:1.

As will be understood, details and particulars concerning hybridization of the nucleotide sequences will be the same or analogous to the hybridization aspects discussed in the section entitled “Polypeptides Having Cellobiase Activity” herein.

Nucleic Acid Constructs

The present invention also relates to nucleic acid constructs comprising a nucleotide sequence of the present invention operably linked to one or more control sequences that direct the expression of the coding sequence in a suitable host cell under conditions compatible with the control sequences.

A nucleotide sequence encoding a polypeptide of the present invention may be manipulated in a variety of ways to provide for expression of the polypeptide. Manipulation of the nucleotide sequence prior to its insertion into a vector may be desirable or necessary depending on the expression vector. The techniques for modifying nucleotide sequences utilizing recombinant DNA methods are well known in the art.

The control sequence may be an appropriate promoter sequence, a nucleotide sequence which is recognized by a host cell for expression of the nucleotide sequence. The promoter sequence contains transcriptional control sequences, which mediate the expression of the polypeptide. The promoter may be any nucleotide sequence which shows transcriptional activity in the host cell of choice including mutant, truncated, and hybrid promoters, and may be obtained from genes encoding extracellular or intracellular polypeptides either homologous or heterologous to the host cell.

Examples of suitable promoters for directing the transcription of the nucleic acid constructs of the present invention, especially in a bacterial host cell, are the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (Villa-Kamaroff et al., 1978, Proceedings of the National Academy of Sciences USA 75: 3727-3731), as well as the tac promoter (DeBoer et al., 1983, Proceedings of the National Academy of Sciences USA 80: 21-25). Further promoters are described in “Useful proteins from recombinant bacteria” in Scientific American, 1980, 242: 74-94; and in Sambrook et al., 1989, supra.

Examples of suitable promoters for directing the transcription of the nucleic acid constructs of the present invention in a filamentous fungal host cell are promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are described by Romanos et al., 1992, Yeast 8: 423-488.

The control sequence may also be a suitable transcription terminator sequence, a sequence recognized by a host cell to terminate transcription. The terminator sequence is operably linked to the 3′ terminus of the nucleotide sequence encoding the polypeptide. Any terminator which is functional in the host cell of choice may be used in the present invention.

Preferred terminators for filamentous fungal host cells are obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be a suitable leader sequence, a nontranslated region of an mRNA which is important for translation by the host cell. The leader sequence is operably linked to the 5′ terminus of the nucleotide sequence encoding the polypeptide. Any leader sequence that is functional in the host cell of choice may be used in the present invention.

Preferred leaders for filamentous fungal host cells are obtained from the genes for Aspergillus oryzae TAKA amylase and Aspergillus nidulans triose phosphate isomerase. Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′ terminus of the nucleotide sequence and which, when transcribed, is recognized by the host cell as a signal to add polyadenosine residues to transcribed mRNA. Any polyadenylation sequence which is functional in the host cell of choice may be used in the present invention.

Preferred polyadenylation sequences for filamentous fungal host cells are obtained from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase.

Useful polyadenylation sequences for yeast host cells are described by Guo and Sherman, 1995, Molecular Cellular Biology 15: 5983-5990.

The control sequence may also be a signal peptide coding region that codes for an amino acid sequence linked to the amino terminus of a polypeptide and directs the encoded polypeptide into the cell's secretory pathway. The 5′ end of the coding sequence of the nucleotide sequence may inherently contain a signal peptide coding region naturally linked in translation reading frame with the segment of the coding region which encodes the secreted polypeptide. Alternatively, the 5′ end of the coding sequence may contain a signal peptide coding region which is foreign to the coding sequence. The foreign signal peptide coding region may be required where the coding sequence does not naturally contain a signal peptide coding region. Alternatively, the foreign signal peptide coding region may simply replace the natural signal peptide coding region in order to enhance secretion of the polypeptide. However, any signal peptide coding region which directs the expressed polypeptide into the secretory pathway of a host cell of choice may be used in the present invention.

The signal peptide coding region is nucleotides 30 to 86 of SEQ ID NO:1 which encode amino acids −19 to −1 of SEQ ID NO:2.

Effective signal peptide coding regions for bacterial host cells are the signal peptide coding regions obtained from the genes for Bacillus NCIB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding regions for filamentous fungal host cells are the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase.

Useful signal peptides for yeast host cells are obtained from the genes for Saccharomyces cerevisiae alpha-factor and Saccharomyces cerevisiae invertase. Other useful signal peptide coding regions are described by Romanos et al., 1992, supra.

The control sequence may also be a propeptide coding region that codes for an amino acid sequence positioned at the amino terminus of a polypeptide. The resultant polypeptide is known as a proenzyme or propolypeptide (or a zymogen in some cases). A propolypeptide is generally inactive and can be converted to a mature active polypeptide by catalytic or autocatalytic cleavage of the propeptide from the propolypeptide. The propeptide coding region may be obtained from the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila laccase (WO 95/33836).

Where both signal peptide and propeptide regions are present at the amino terminus of a polypeptide, the propeptide region is positioned next to the amino terminus of a polypeptide and the signal peptide region is positioned next to the amino terminus of the propeptide region.

It may also be desirable to add regulatory sequences which allow the regulation of the expression of the polypeptide relative to the growth of the host cell. Examples of regulatory systems are those which cause the expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used. In filamentous fungi, the TAKA alpha-amylase promoter, Aspergillus niger glucoamylase promoter, and Aspergillus oryzae glucoamylase promoter may be used as regulatory sequences. Other examples of regulatory sequences are those which allow for gene amplification. In eukaryotic systems, these include the dihydrofolate reductase gene which is amplified in the presence of methotrexate, and the metallothionein genes which are amplified with heavy metals. In these cases, the nucleotide sequence encoding the polypeptide would be operably linked with the regulatory sequence.

Expression Vectors

The present invention also relates to recombinant expression vectors comprising the nucleic acid construct of the invention. The various nucleotide and control sequences described above may be joined together to produce a recombinant expression vector which may include one or more convenient restriction sites to allow for insertion or substitution of the nucleotide sequence encoding the polypeptide at such sites. Alternatively, the nucleotide sequence of the present invention may be expressed by inserting the nucleotide sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) which can be conveniently subjected to recombinant DNA procedures and can bring about the expression of the nucleotide sequence. The choice of the vector will typically depend on the compatibility of the vector with the host cell into which the vector is to be introduced. The vectors may be linear or closed circular plasmids.

The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome.

The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

The vectors of the present invention preferably contain one or more selectable markers which permit easy selection of transformed cells. A selectable marker is a gene the product of which provides for biocide or viral resistance, resistance to heavy metals, prototrophy to auxotrophs, and the like.

Examples of bacterial selectable markers are the dal genes from Bacillus subtilis or Bacillus licheniformis, or markers which confer antibiotic resistance such as ampicillin, kanamycin, chloramphenicol or tetracycline resistance. Suitable markers for yeast host cells are ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hygB (hygromycin phosphotransferase), niaD (nitrate reductase), pyrG (orotidine-5′-phosphate decarboxylase), sC (sulfate adenyltransferase), trpC (anthranilate synthase), as well as equivalents thereof.

Preferred for use in an Aspergillus cell are the amdS and pyrG genes of Aspergillus nidulans or Aspergillus oryzae and the bar gene of Streptomyces hygroscopicus.

The vectors of the present invention preferably contain an element(s) that permits stable integration of the vector into the host cell's genome or autonomous replication of the vector in the cell independent of the genome.

For integration into the host cell genome, the vector may rely on the nucleotide sequence encoding the polypeptide or any other element of the vector for stable integration of the vector into the genome by homologous or nonhomologous recombination. Alternatively, the vector may contain additional nucleotide sequences for directing integration by homologous recombination into the genome of the host cell. The additional nucleotide sequences enable the vector to be integrated into the host cell genome at a precise location(s) in the chromosome(s). To increase the likelihood of integration at a precise location, the integrational elements should preferably contain a sufficient number of nucleotides, such as 100 to 1,500 base pairs, preferably 400 to 1,500 base pairs, and most preferably 800 to 1,500 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleotide sequences. On the other hand, the vector may be integrated into the genome of the host cell by non-homologous recombination.

For autonomous replication, the vector may further comprise an origin of replication enabling the vector to replicate autonomously in the host cell in question. Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus. Examples of origins of replication for use in a yeast host cell are the 2 micron origin of replication, ARS1, ARS4, the combination of ARS1 and CEN3, and the combination of ARS4 and CEN6. The origin of replication may be one having a mutation which makes its functioning temperature-sensitive in the host cell (see, e.g., Ehrlich, 1978, Proceedings of the National Academy of Sciences USA 75: 1433).

More than one copy of a nucleotide sequence of the present invention may be inserted into the host cell to increase production of the gene product. An increase in the copy number of the nucleotide sequence can be obtained by integrating at least one additional copy of the sequence into the host cell genome or by including an amplifiable selectable marker gene with the nucleotide sequence where cells containing amplified copies of the selectable marker gene, and thereby additional copies of the nucleotide sequence, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

Host Cells

The present invention also relates to recombinant a host cell comprising the nucleic acid construct of the invention, which are advantageously used in the recombinant production of the polypeptides. A vector comprising a nucleotide sequence of the present invention is introduced into a host cell so that the vector is maintained as a chromosomal integrant or as a self-replicating extra-chromosomal vector as described earlier.

The host cell may be a unicellular microorganism, e.g., a prokaryote, or a non-unicellular microorganism, e.g., a eukaryote.

Useful unicellular cells are bacterial cells such as gram positive bacteria including, but not limited to, a Bacillus cell, e.g., Bacillus alkalophilus, Bacillus amyloliquefaciens, Bacillus brevis, Bacillus circulans, Bacillus clausii, Bacillus coagulans, Bacillus lautus, Bacillus lentus, Bacillus licheniformis, Bacillus megaterium, Bacillus stearothermophilus, Bacillus subtilis, and Bacillus thuringiensis; or a Streptomyces cell, e.g., Streptomyces lividans or Streptomyces murinus, or gram negative bacteria such as E. coli and Pseudomonas sp. In a preferred embodiment, the bacterial host cell is a Bacillus lentus, Bacillus licheniformis, Bacillus stearothermophilus, or Bacillus subtilis cell. In another preferred embodiment, the Bacillus cell is an alkalophilic Bacillus.

The introduction of a vector into a bacterial host cell may, for instance, be effected by protoplast transformation (see, e.g., Chang and Cohen, 1979, Molecular General Genetics 168: 111-115), using competent cells (see, e.g., Young and Spizizin, 1961, Journal of Bacteriology 81: 823-829, or Dubnau and Davidoff-Abelson, 1971, Journal of Molecular Biology 56: 209-221), electroporation (see, e.g., Shigekawa and Dower, 1988, Biotechniques 6: 742-751), or conjugation (see, e.g., Koehler and Thorne, 1987, Journal of Bacteriology 169: 5771-5278).

The host cell may be a eukaryote, such as a mammalian, insect, plant, or fungal cell.

In a preferred embodiment, the host cell is a fungal cell. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra).

In a more preferred embodiment, the fungal host cell is a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacteriol. Symposium Series No. 9, 1980).

In an even more preferred embodiment, the yeast host cell is a Candida, Hansenula, Kluyveromyces, Pichia, Saccharomyces, Schizosaccharomyces, or Yarrowia cell.

In a most preferred embodiment, the yeast host cell is a Saccharomyces carlsbergensis, Saccharomyces cerevisiae, Saccharomyces diastaticus, Saccharomyces douglasii, Saccharomyces kluyveri, Saccharomyces norbensis or Saccharomyces oviformis cell. In another most preferred embodiment, the yeast host cell is a Kluyveromyces lactis cell. In another most preferred embodiment, the yeast host cell is a Yarrowia lipolytica cell.

In another more preferred embodiment, the fungal host cell is a filamentous fungal cell. “Filamentous fungi” include all filamentous forms of the subdivision Eumycota and Oomycota (as defined by Hawksworth et al., 1995, supra). The filamentous fungi are characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

In an even more preferred embodiment, the filamentous fungal host cell is a cell of a species of, but not limited to, Acremonium, Aspergillus, Fusarium, Humicola, Mucor, Myceliophthora, Neurospora, Penicillium, Thielavia, Tolypocladium, or Trichoderma.

In a most preferred embodiment, the filamentous fungal host cell is an Aspergillus awamori, Aspergillus foetidus, Aspergillus japonicus, Aspergillus nidulans, Aspergillus niger or Aspergillus oryzae cell. In another most preferred embodiment, the filamentous fungal host cell is a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, or Fusarium venenatum cell. In an even most preferred embodiment, the filamentous fungal parent cell is a Fusarium venenatum (Nirenberg sp. nov.) cell. In another most preferred embodiment, the filamentous fungal host cell is a Humicola insolens, Humicola lanuginosa, Mucor miehei, Myceliophthora thermophila, Neurospora crassa, Penicillium purpurogenum, Thielavia terrestris, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus host cells are described in EP 238 023 and Yelton et al., 1984, Proceedings of the National Academy of Sciences USA 81: 1470-1474. Suitable methods for transforming Fusarium species are described by Malardier et al., 1989, Gene 78: 147-156 and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75: 1920.

Methods of Production

The present invention also relates to methods for producing a polypeptide of the present invention comprising (a) cultivating a strain, which in its wild-type form is capable of producing the polypeptide; and (b) recovering the polypeptide. Preferably, the strain is of the genus Aspergillus, and more preferably Aspergillus oryzae.

The present invention also relates to methods for producing a polypeptide of the present invention comprising (a) cultivating a host cell under conditions conducive for production of the polypeptide; and (b) recovering the polypeptide.

In the production methods of the present invention, the cells are cultivated in a nutrient medium suitable for production of the polypeptide using methods known in the art. For example, the cell may be cultivated by shake flask cultivation, small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptides may be detected using methods known in the art that are specific for the polypeptides. These detection methods may include use of specific antibodies, formation of an enzyme product, or disappearance of an enzyme substrate. For example, an enzyme assay may be used to determine the activity of the polypeptide as described herein.

The resulting polypeptide may be recovered by methods known in the art. For example, the polypeptide may be recovered from the nutrient medium by conventional procedures including, but not limited to, centrifugation, filtration, extraction, spray-drying, evaporation, or precipitation.

The polypeptides of the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989).

Production of Ethanol from Biomass

Ethanol can be produced by enzymatic degradation of biomass and conversion of the released polysaccharides to ethanol. This kind of ethanol is often referred to as bioethanol or biofuel. It can be used as a fuel additive or extender in blends of from less than 1% and up to 100% (a fuel substitute). In some countries, such as Brazil, ethanol is substituting gasoline to a very large extent.

The predominant polysaccharide in the primary cell wall of biomass is cellulose, the second most abundant is hemi-cellulose, and the third is pectin. The secondary cell wall, produced after the cell has stopped growing, also contains polysaccharides and is strengthened through polymeric lignin covalently cross-linked to hemicellulose. Cellulose is a homopolymer of anhydrocellobiose and thus a linear beta-(1-4)-D-glucan, while hemicelluloses include a variety of compounds, such as xylans, xyloglucans, arabinoxylans, and mannans in complex branched structures with a spectrum of substituents. Although generally polymorphous, cellulose is found in plant tissue primarily as an insoluble crystalline matrix of parallel glucan chains. Hemicelluloses usually hydrogen bond to cellulose, as well as to other hemicelluloses, which helps stabilize the cell wall matrix.

Three major classes of cellulase enzymes are used to breakdown biomass:
The “endo-1,4-beta-glucanases” or 1,4-beta-D-glucan-4-glucanohydrolases (EC 3.2.1.4), which act randomly on soluble and insoluble 1,4-beta-glucan substrates.
The “exo-1,4-beta-D-glucanases” including both the 1,4-beta-D-glucan glucohydrolases (EC 3.2.1.74), which liberate D-glucose from 1,4-beta-D-glucans and hydrolyze D-cellobiose slowly, and 1,4-beta-D-glucan cellobiohydrolase (EC 3.2.1.91), also referred to as cellobiohydrolase I, which liberates D-cellobiose from 1,4-beta-glucans.
The “beta-D-glucosidases” or beta-D-glucoside glucohydrolases (EC 3.2.1.21), which act to release D-glucose units from cellobiose and soluble cellodextrins, as well as an array of glycosides.

These three classes of enzymes work together synergistically in a complex interplay that results in efficient decrystallization and hydrolysis of native cellulose from biomass to yield the reducing sugars which are converted to ethanol by fermentation.

Accordingly the present invention also relates to a method for producing ethanol from biomass, comprising contacting the biomass with the polypeptide according to the invention, as well to a use of the polypeptide according to the invention for producing ethanol.

Plants

The present invention also relates to a transgenic plant, plant part, or plant cell which has been transformed with a nucleotide sequence encoding a polypeptide having cellobiase activity of the present invention so as to express and produce the polypeptide in recoverable quantities. The polypeptide may be recovered from the plant or plant part. Alternatively, the plant or plant part containing the recombinant polypeptide may be used as such for improving the quality of a food or feed, e.g., improving nutritional value, palatability, and rheological properties, or to destroy an antinutritive factor.

The transgenic plant can be dicotyledonous (a dicot) or monocotyledonous (a monocot). Examples of monocot plants are grasses, such as meadow grass (blue grass, Poa), forage grass such as Festuca, Lolium, temperate grass, such as Agrostis, and cereals, e.g., wheat, oats, rye, barley, rice, sorghum, and maize (corn).

Examples of dicot plants are tobacco, potato, sugar beet, legumes, such as lupins, pea, bean and soybean, and cruciferous plants (family Brassicaceae), such as cauliflower, rape seed, and the closely related model organism Arabidopsis thaliana.

Examples of plant parts are stem, callus, leaves, root, fruits, seeds, and tubers. Also specific plant tissues, such as chloroplast, apoplast, mitochondria, vacuole, peroxisomes, and cytoplasm are considered to be a plant part. Furthermore, any plant cell, whatever the tissue origin, is considered to be a plant part.

Also included within the scope of the present invention are the progeny of such plants, plant parts and plant cells.

The transgenic plant or plant cell expressing a polypeptide of the present invention may be constructed in accordance with methods known in the art. Briefly, the plant or plant cell is constructed by incorporating one or more expression constructs encoding a polypeptide of the present invention into the plant host genome and propagating the resulting modified plant or plant cell into a transgenic plant or plant cell.

Conveniently, the expression construct is a nucleic acid construct which comprises a nucleotide sequence encoding a polypeptide of the present invention operably linked with appropriate regulatory sequences required for expression of the nucleotide sequence in the plant or plant part of choice. Furthermore, the expression construct may comprise a selectable marker useful for identifying host cells into which the expression construct has been integrated and DNA sequences necessary for introduction of the construct into the plant in question (the latter depends on the DNA introduction method to be used).

The choice of regulatory sequences, such as promoter and terminator sequences and optionally signal or transit sequences is determined, for example, on the basis of when, where, and how the polypeptide is desired to be expressed. For instance, the expression of the gene encoding a polypeptide of the present invention may be constitutive or inducible, or may be developmental, stage or tissue specific, and the gene product may be targeted to a specific tissue or plant part such as seeds or leaves. Regulatory sequences are, for example, described by Tague et al., 1988, Plant Physiology 86: 506.

For constitutive expression, the ³⁵S-CaMV promoter may be used (Franck et al., 1980, Cell 21: 285-294). Organ-specific promoters may be, for example, a promoter from storage sink tissues such as seeds, potato tubers, and fruits (Edwards & Coruzzi, 1990, Ann. Rev. Genet. 24: 275-303), or from metabolic sink tissues such as meristems (Ito et al., 1994, Plant Mol. Biol. 24: 863-878), a seed specific promoter such as the glutelin, prolamin, globulin, or albumin promoter from rice (Wu et al., 1998, Plant and Cell Physiology 39: 885-889), a Vicia faba promoter from the legumin B4 and the unknown seed protein gene from Vicia faba (Conrad et al., 1998, Journal of Plant Physiology 152: 708-711), a promoter from a seed oil body protein (Chen et al., 1998, Plant and Cell Physiology 39: 935-941), the storage protein napA promoter from Brassica napus, or any other seed specific promoter known in the art, e.g., as described in WO 91/14772. Furthermore, the promoter may be a leaf specific promoter such as the rbcs promoter from rice or tomato (Kyozuka et al., 1993, Plant Physiology 102: 991-1000, the chlorella virus adenine methyltransferase gene promoter (Mitra and Higgins, 1994, Plant Molecular Biology 26: 85-93), or the aldP gene promoter from rice (Kagaya et al., 1995, Molecular and General Genetics 248: 668-674), or a wound inducible promoter such as the potato pin2 promoter (Xu et al., 1993, Plant Molecular Biology 22: 573-588).

A promoter enhancer element may also be used to achieve higher expression of the enzyme in the plant. For instance, the promoter enhancer element may be an intron which is placed between the promoter and the nucleotide sequence encoding a polypeptide of the present invention. For instance, Xu et al., 1993, supra disclose the use of the first intron of the rice actin 1 gene to enhance expression.

The selectable marker gene and any other parts of the expression construct may be chosen from those available in the art.

The nucleic acid construct is incorporated into the plant genome according to conventional techniques known in the art, including Agrobacterium-mediated transformation, virus-mediated transformation, microinjection, particle bombardment, biolistic transformation, and electroporation (Gasser et al., 1990, Science 244: 1293; Potrykus, 1990, Bio/Technology 8: 535; Shimamoto et al., 1989, Nature 338: 274).

Presently, Agrobacterium tumefaciens-mediated gene transfer is the method of choice for generating transgenic dicots (for a review, see Hooykas and Schilperoort, 1992, Plant Molecular Biology 19: 15-38). However it can also be used for transforming monocots, although other transformation methods are generally preferred for these plants. Presently, the method of choice for generating transgenic monocots is particle bombardment (microscopic gold or tungsten particles coated with the transforming DNA) of embryonic calli or developing embryos (Christou, 1992, Plant Journal 2: 275-281; Shimamoto, 1994, Current Opinion Biotechnology 5: 158-162; Vasil et al., 1992, Bio/Technology 10: 667-674). An alternative method for transformation of monocots is based on protoplast transformation as described by Omirulleh et al., 1993, Plant Molecular Biology 21: 415-428.

Following transformation, the transformants having incorporated therein the expression construct are selected and regenerated into whole plants according to methods well-known in the art.

The present invention also relates to methods for producing a polypeptide of the present invention comprising (a) cultivating a transgenic plant or a plant cell comprising a nucleotide sequence encoding a polypeptide having cellobiase activity of the present invention under conditions conducive for production of the polypeptide; and (b) recovering the polypeptide.

Compositions

In a still further aspect, the present invention relates to compositions comprising a polypeptide of the present invention.

The composition may comprise a polypeptide of the invention as the major enzymatic component, e.g., a mono-component composition. Alternatively, the composition may comprise multiple enzymatic activities, such as an aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, invertase, laccase, lipase, mannosidase, oxidase, pectinolytic enzyme, peptidoglutaminase, peroxidase, phytase, polyphenoloxidase, proteolytic enzyme, ribonuclease, transglutaminase, or xylanase.

The compositions may be prepared in accordance with methods known in the art and may be in the form of a liquid or a dry composition. For instance, the polypeptide composition may be in the form of a granulate or a microgranulate. The polypeptide to be included in the composition may be stabilized in accordance with methods known in the art.

Examples are given below of preferred uses of the polypeptide compositions of the invention. The dosage of the polypeptide composition of the invention and other conditions under which the composition is used may be determined on the basis of methods known in the art.

Detergent Composition

The cellobiase of the invention may be added to and thus become a component of a detergent composition.

The detergent composition of the invention may for example be formulated as a hand or machine laundry detergent composition including a laundry additive composition suitable for pre-treatment of stained fabrics and a rinse added fabric softener composition, or be formulated as a detergent composition for use in general household hard surface cleaning operations, or be formulated for hand or machine dishwashing operations.

In a specific aspect, the invention provides a detergent additive comprising the cellobiase of the invention. The detergent additive as well as the detergent composition may comprise one or more other enzymes such as a protease, a lipase, a cutinase, an amylase, a carbohydrase, a cellulase, a pectinase, a mannanase, an arabinase, a galactanase, a xylanase, an oxidase, e.g., a laccase, and/or a peroxidase.

In general the properties of the chosen enzyme(s) should be compatible with the selected detergent, (i.e. pH-optimum, compatibility with other enzymatic and non-enzymatic ingredients, etc.), and the enzyme(s) should be present in effective amounts.

Proteases: Suitable proteases include those of animal, vegetable or microbial origin. Microbial origin is preferred. Chemically modified or protein engineered mutants are included. The protease may be a serine protease or a metallo protease, preferably an alkaline microbial protease or a trypsin-like protease. Examples of alkaline proteases are subtilisins, especially those derived from Bacillus, e.g., subtilisin Novo, subtilisin Carlsberg, subtilisin 309, subtilisin 147 and subtilisin 168 (described in WO 89/06279). Examples of trypsin-like proteases are trypsin (e.g. of porcine or bovine origin) and the Fusarium protease described in WO 89/06270 and WO 94/25583.

Examples of useful proteases are the variants described in WO 92/19729, WO 98/20115, WO 98/20116, and WO 98/34946, especially the variants with substitutions in one or more of the following positions: 27, 36, 57, 76, 87, 97, 101, 104, 120, 123, 167, 170, 194, 206, 218, 222, 224, 235 and 274.

Preferred commercially available protease enzymes include Alcalase™, Savinase™, Primase™, Everlase™, Esperase™, and Kannase™ (Novozymes A/S), Maxatase™, Maxacal™, Maxapem™, Properase™, Purafect™, Purafect OXP™, FN2™, and FN3™ (Genencor International Inc.).

Lipases: Suitable lipases include those of bacterial or fungal origin. Chemically modified or protein engineered mutants are included. Examples of useful lipases include lipases from Humicola (synonym Thermomyces), e.g. from H. lanuginosa (T. lanuginosus) as described in EP 258 068 and EP 305 216 or from H. insolens as described in WO 96/13580, a Pseudomonas lipase, e.g. from P. alcaligenes or P. pseudoalcaligenes (EP 218 272), P. cepacia (EP 331 376), P. stutzeri (GB 1,372,034), P. fluorescens, Pseudomonas sp. strain SD 705 (WO 95/06720 and WO 96/27002), P. wisconsinensis (WO 96/12012), a Bacillus lipase, e.g. from B. subtilis (Dartois et al. (1993), Biochemica et Biophysica Acta, 1131, 253-360), B. stearothermophilus (JP 64/744992) or B. pumilus (WO 91/16422).

Other examples are lipase variants such as those described in WO 92/05249, WO 94/01541, EP 407 225, EP 260 105, WO 95/35381, WO 96/00292, WO 95/30744, WO 94/25578, WO 95/14783, WO 95/22615, WO 97/04079 and WO 97/07202.

Preferred commercially available lipase enzymes include Lipolase™ and Lipolase Ultra™ (Novozymes A/S).

Amylases: Suitable amylases (α and/or β) include those of bacterial or fungal origin. Chemically modified or protein engineered mutants are included. Amylases include, for example, α-amylases obtained from Bacillus, e.g. a special strain of B. licheniformis, described in more detail in GB 1,296,839.

Examples of useful amylases are the variants described in WO 94/02597, WO 94/18314, WO 96/23873, and WO 97/43424, especially the variants with substitutions in one or more of the following positions: 15, 23, 105, 106, 124, 128, 133, 154, 156, 181, 188, 190, 197, 202, 208, 209, 243, 264, 304, 305, 391, 408, and 444.

Commercially available amylases are Duramyl™, Termamyl™, Fungamyl™ and BAN™ (Novozymes A/S), Rapidase™ and Purastar™ (from Genencor International Inc.).

Cellulases: Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g. the fungal cellulases produced from Humicola insolens, Myceliophthora thermophila and Fusarium oxysporum disclosed in U.S. Pat. No. 4,435,307, U.S. Pat. No. 5,648,263, U.S. Pat. No. 5,691,178, U.S. Pat. No. 5,776,757 and WO 89/09259.

Especially suitable cellulases are the alkaline or neutral cellulases having colour care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. No. 5,457,046, U.S. Pat. No. 5,686,593, U.S. Pat. No. 5,763,254, WO 95/24471, WO 98/12307 and PCT/DK98/00299.

Commercially available cellulases include Celluzyme™, and Carezyme™ (Novozymes A/S), Clazinase™, and Puradax HA™ (Genencor International Inc.), and KAC-500(B)™ (Kao Corporation).

Peroxidases/Oxidases: Suitable peroxidases/oxidases include those of plant, bacterial or fungal origin. Chemically modified or protein engineered mutants are included. Examples of useful peroxidases include peroxidases from Coprinus, e.g. from C. cinereus, and variants thereof as those described in WO 93/24618, WO 95/10602, and WO 98/15257.

The detergent enzyme(s) may be included in a detergent composition by adding separate additives containing one or more enzymes, or by adding a combined additive comprising all of these enzymes. A detergent additive of the invention, i.e. a separate additive or a combined additive, can be formulated e.g. as a granulate, a liquid, a slurry, etc. Preferred detergent additive formulations are granulates, in particular non-dusting granulates, liquids, in particular stabilized liquids, or slurries.

Non-dusting granulates may be produced, e.g., as disclosed in U.S. Pat. Nos. 4,106,991 and 4,661,452 and may optionally be coated by methods known in the art. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238,216.

The detergent composition of the invention may be in any convenient form, e.g., a bar, a tablet, a powder, a granule, a paste or a liquid. A liquid detergent may be aqueous, typically containing up to 70% water and 0-30% organic solvent, or non-aqueous.

The detergent composition comprises one or more surfactants, which may be non-ionic including semi-polar and/or anionic and/or cationic and/or zwitterionic. The surfactants are typically present at a level of from 0.1% to 60% by weight.

When included therein the detergent will usually contain from about 1% to about 40% of an anionic surfactant such as linear alkylbenzenesulfonate, alpha-olefinsulfonate, alkyl sulfate (fatty alcohol sulfate), alcohol ethoxysulfate, secondary alkanesulfonate, alpha-sulfo fatty acid methyl ester, alkyl- or alkenylsuccinic acid or soap.

When included therein the detergent will usually contain from about 0.2% to about 40% of a non-ionic surfactant such as alcohol ethoxylate, nonylphenol ethoxylate, alkylpolyglycoside, alkyldimethylamineoxide, ethoxylated fatty acid monoethanolamide, fatty acid monoethanolamide, polyhydroxy alkyl fatty acid amide, or N-acyl N-alkyl derivatives of glucosamine (“glucamides”).

The detergent may contain 0-65% of a detergent builder or complexing agent such as zeolite, diphosphate, triphosphate, phosphonate, carbonate, citrate, nitrilotriacetic acid, ethylenediaminetetraacetic acid, diethylenetriaminepentaacetic acid, alkyl- or alkenylsuccinic acid, soluble silicates or layered silicates (e.g. SKS-6 from Hoechst).

The detergent may comprise one or more polymers. Examples are carboxymethylcellulose, poly(vinylpyrrolidone), poly (ethylene glycol), poly(vinyl alcohol), poly(vinylpyridine-N-oxide), poly(vinylimidazole), polycarboxylates such as polyacrylates, maleic/acrylic acid copolymers and lauryl methacrylate/acrylic acid copolymers.

The detergent may contain a bleaching system, which may comprise a H2O2 source such as perborate or percarbonate which may be combined with a peracid-forming bleach activator such as tetraacetylethylenediamine or nonanoyloxybenzenesulfonate. Alternatively, the bleaching system may comprise peroxyacids of e.g. the amide, imide, or sulfone type.

The enzyme(s) of the detergent composition of the invention may be stabilized using conventional stabilizing agents, e.g., a polyol such as propylene glycol or glycerol, a sugar or sugar alcohol, lactic acid, boric acid, or a boric acid derivative, e.g., an aromatic borate ester, or a phenyl boronic acid derivative such as 4-formylphenyl boronic acid, and the composition may be formulated as described in e.g. WO 92/19709 and WO 92/19708.

The detergent may also contain other conventional detergent ingredients such as e.g. fabric conditioners including clays, foam boosters, suds suppressors, anti-corrosion agents, soil-suspending agents, anti-soil redeposition agents, dyes, bactericides, optical brighteners, hydrotropes, tarnish inhibitors, or perfumes.

It is at present contemplated that in the detergent compositions any enzyme, in particular the cellobiase of the invention, may be added in an amount corresponding to 0.01-100 mg of enzyme protein per liter of wash liqour, preferably 0.05-5 mg of enzyme protein per liter of wash liquor, in particular 0.1-1 mg of enzyme protein per liter of wash liquor.

The cellobiase of the invention may additionally be incorporated in the detergent formulations disclosed in WO 97/07202, which is hereby incorporated as reference.

DNA Recombination (Shuffling)

The nucleotide sequence of SEQ ID NO:1 may be used in a DNA recombination (or shuffling) process. The new polynucleotide sequences obtained in such a process may encode new polypeptides having cellobiase activity with improved properties, such as improved stability (storage stability, thermostability), improved specific activity, improved pH-optimum, and/or improved tolerance towards specific compounds.

Shuffling between two or more homologous input polynucleotides (starting-point polynucleotides) involves fragmenting the polynucleotides and recombining the fragments, to obtain output polynucleotides (i.e. polynucleotides that have been subjected to a shuffling cycle) wherein a number of nucleotide fragments are exchanged in comparison to the input polynucleotides.

DNA recombination or shuffling may be a (partially) random process in which a library of chimeric genes is generated from two or more starting genes. A number of known formats can be used to carry out this shuffling or recombination process.

The process may involve random fragmentation of parental DNA followed by reassembly by PCR to new full-length genes, e.g. as presented in U.S. Pat. No. 5,605,793, U.S. Pat. No. 5,811,238, U.S. Pat. No. 5,830,721, U.S. Pat. No. 6,117,679. In-vitro recombination of genes may be carried out, e.g. as described in U.S. Pat. No. 6,159,687, WO98/41623, U.S. Pat. No. 6,159,688, U.S. Pat. No. 5,965,408, U.S. Pat. No. 6,153,510. The recombination process may take place in vivo in a living cell, e.g. as described in WO 97/07205 and WO 98/28416.

The parental DNA may be fragmented by DNA'se I treatment or by restriction endonuclease digests as descriobed by Kikuchi et al (2000a, Gene 236:159-167). Shuffling of two parents may be done by shuffling single stranded parental DNA of the two parents as described in Kikuchi et al (2000b, Gene 243:133-137).

A particular method of shuffling is to follow the methods described in Crameri et al, 1998, Nature, 391: 288-291 and Ness et al. Nature Biotechnology 17: 893-896. Another format would be the methods described in U.S. Pat. No. 6,159,687: Examples 1 and 2.

The present invention is further described by the following examples which should not be construed as limiting the scope of the invention.

### EXAMPLES

Chemicals used as buffers and substrates were commercial products of at least reagent grade.

Cellobiase Assay

PNP Glucose with Stop Reagent

Substrate solution: 5 mM PNP beta-D-Glucose (Sigma N-7006) substrate in 0.1 M Na-acetate buffer, pH 4.0;
Stop reagent: 0.1 M Na-carbonate, pH 11.5.

50 μl cellobiase solution is mixed with 1 ml substrate solution and incubated 20 minutes at 40° C. The reaction is stopped by addition of 5 ml stop reagent. Absorbance is measured at 404 nm.

Degradation of Cellobiose (CBU Analysis)

Cellobiase hydrolyzes beta-1,4 bonds in cellobiose to release two glucose molecules. The amount of glucose released is determined using a suitable glucose analysis method, such as the hexokinase method (e.g. Bergmeyer et al.: D-glucose. Determination with hexokinase and glucose-6-phosphatase dehydrogenase. In: Bergmeyer H U, Gawehn K, eds. Methods of Enzymatic Analysis. New York: Academic Press; 1974:196-201; or Kunst, A., Draeger, B. & Ziegenhorn, J. (1984) in Methods of Enzymatic Analysis (Bergmeyer, H U, ed), 3rd Ed, Vol. 6, pp. 163-172, VCH, Weinheim, W. Germany-Deer-field) which is commercially available from Roche (#127183). One cellobiase unit (CBU) is the amount of enzyme which releases 2 μmol glucose per minute at 40° C., pH 5 with cellobiose as substrate.

The cellobiase sample is diluted in 0.1 M acetate buffer, pH 5.0 to approx. 0.05-0.25 CBU/ml. 0.5 ml of this solution is mixed with 2.5 ml cellobiose substrate solution (0.2 g D-(+)cellobiose, Sigma C-7252, in 0.1 M acetate buffer, pH 5.0) and heated at 40° C. for 15 min. The reaction is stopped by adding 300 μl 1N perchloric acid and the released glucose is measured.

The specific activity of Aspergillus oryzae cellobiase is 150 CBU pr. mg protein, and the specific activity of Aspergillus niger cellobiase is 10 CBU pr. mg protein.

Materials

Strains

BECh2: Construction of this strain is described in WO 00/39322;
JaL406: Construction of this strain is described in Example 2.
   Plasmids
pYES 2.0: Available from Invitrogen Corporation, San Diego, Calif., USA;
pJaL621: Construction of this plasmid is described in Example 1;
pJaL660: Construction of this plasmid is described in Example 2;
pMT2188: Construction of this plasmid is described in Example 2;
pCaHj527: Construction of this plasmid is described in WO 00/70064.

### Example 1

Cloning of an Aspergillus oryzae Cellobiase (Beta-Glucosidase)

Construction of a Directional cDNA Library from Aspergillus oryzae Strain IF04177

The Aspergillus oryzae strain IF04177 was grown in a 20-litre lab fermentor in 10-litre scale using urea and yeast extract as nitrogen sources, dextrose as carbon source in the batch medium, and nutriose (maltose syrup) as carbon source in the feed. The composition of the growth medium in the fermentor was (g/l): dextrose 27.5, yeast extract 5.0, MgSO4-7H2O 2.0, K2SO4 3.0, KH2PO4 2.0, citric acid 4.0, urea 5.0, and trace elements 0.5 ml/l. The fungal mycelium was harvested after 68.3 hours of growth at 30° C., immediately frozen in liquid N2, and stored at −80° C.

Extraction of Total RNA

Total RNA was prepared by extraction with guanidinium thiocyanate followed by ultracentrifugation through a 5.7 M CsCl cushion (Chirgwin et al., 1979 Biochemistry 18: 5294-5299) using the following modifications. The frozen mycelia was ground in liquid N2 to fine powder with a mortar and a pestle, followed by grinding in a precooled coffee mill, and immediately suspended in 5 volumes of RNA extraction buffer (4 M GuSCN, 0.5% Na-laurylsarcosine, 25 mM Na-citrate, pH 7.0, 0.1 M β-mercaptoethanol). The mixture was stirred for 30 min. at room temperature and centrifuged (20 min., 10000 rpm, Beckman) to pellet the cell debris. The supernatant was collected, carefully layered onto a 5.7 M CsCl cushion (5.7 M CsCl, 10 mM EDTA, pH 7.5, 0.1% DEPC; autoclaved prior to use) using 26.5 ml supernatant per 12.0 ml CsCl cushion, and centrifuged to obtain the total RNA (Beckman, SW28 rotor, 25000 rpm, room temperature, 24 hours). After centrifugation the supernatant was carefully removed and the bottom of the tube containing the RNA pellet was cut off and rinsed with 70% EtOH. The total RNA pellet was transferred into an Eppendorf tube, suspended in 500 ml TE, pH 7.6 (if difficult, heat occasionally for 5 min at 65° C.), phenol extracted and precipitated with ethanol for 12 hours at −20° C. (2.5 volumes EtOH, 0.1 volumes 3M NaAc, pH 5.2). The RNA was collected by centrifugation, washed in 70% EtOH, and resuspended in a minimum volume of DEPC-DIW. The RNA concentration was determined by measuring OD260/280.

Isolation of Poly(A)+RNA

The poly(A)+ RNA was isolated by oligo(dT)-cellulose affinity chromatography (Aviv & Leder, 1972 Proc. Natl. Acad. Sci. U.S.A. 69: 1408-1412). Typically, 0.2 g of oligo(dT) cellulose (Boehringer Mannheim) was preswollen in 10 ml of 1× column loading buffer (20 mM Tris-Cl, pH 7.6, 0.5 M NaCl, 1 mM EDTA, 0.1% SDS), loaded onto a DEPC-treated, plugged plastic column (Poly Prep Chromatography Column, Bio Rad), and equilibrated with 20 ml 1× loading buffer. The total RNA (1-2 mg) was heated at 65° C. for 8 min., quenched on ice for 5 min, and after addition of 1 volume 2× column loading buffer to the RNA sample loaded onto the column. The eluate was collected and reloaded 2-3 times by heating the sample as above and quenching on ice prior to each loading. The oligo(dT) column was washed with 10 volumes of 1× loading buffer, then with 3 volumes of medium salt buffer (20 mM Tris-Cl, pH 7.6, 0.1 M NaCl, 1 mM EDTA, 0.1% SDS), followed by elution of the poly(A)+ RNA with 3 volumes of elution buffer (10 mM Tris-Cl, pH 7.6, 1 mM EDTA, 0.05% SDS) preheated to +65° C., by collecting 500 μl fractions. The OD260 was read for each collected fraction, and the mRNA containing fractions were pooled and ethanol precipitated at −20° C. for 12 hours. The poly(A)+ RNA was collected by centrifugation, resuspended in DEPC-DIW and stored in 5-10 μg aliquots at −80° C.

cDNA Synthesis

Double-stranded cDNA was synthesized from 5 μg of Aspergillus oryzae A1560 poly(A)+ RNA by the RNase H method (Gubler & Hoffman 1983 Gene 25: 263-269, Sambrook et al., 1989 Molecular Cloning: A Laboratory Manual. Cold Spring Harbor Lab., Cold Spring Harbor, N.Y.) using the hair-pin modification developed by F. S. Hagen (personal communication). The poly(A)+ RNA (5 μg in 5 μl of DEPC-treated water) was heated at 70° C. for 8 min. in a pre-siliconized, RNase-free Eppendorph tube, quenched on ice, and combined in a final volume of 50 μl with reverse transcriptase buffer (50 mM Tris-Cl, pH 8.3, 75 mM KCl, 3 mM MgCl2, 10 mM DTT, Bethesda Research Laboratories) containing 1 mM of dATP, dGTP and dTTP, and 0.5 mM of 5-methyl-dCTP (Pharmacia), 40 units of human placental ribonuclease inhibitor (RNasin, Promega), 4.81 μg of oligo(dT)18-Not I primer (Pharmacia) and 1000 units of SuperScript II RNase H reverse transcriptase (Bethesda Research Laboratories). First-strand cDNA was synthesized by incubating the reaction mixture at 45° C. for 1 hour. After synthesis, the mRNA:cDNA hybrid mixture was gel filtrated through a MicroSpin S-400 HR (Pharmacia) spin column according to the manufacturers instructions.

After the gel filtration, the hybrids were diluted in 250 μl of second strand buffer (20 mM Tris-Cl, pH 7.4, 90 mM KCl, 4.6 mM MgCl2, 10 mM (NH4)2SO4, 0.16 mM βNAD+) containing 200 μM of each dNTP, 60 units of E. coli DNA polymerase I (Pharmacia), 5.25 units of RNase H (Promega) and 15 units of E. coli DNA ligase (Boehringer Mannheim). Second strand cDNA synthesis was performed by incubating the reaction tube at 16° C. for 2 hours, and an additional 15 min at 25° C. The reaction was stopped by addition of EDTA to 20 mM final concentration followed by phenol and chloroform extractions.

The double-stranded (ds) cDNA was ethanol precipitated at −20° C. for 12 hours by addition of 2 volumes of 96% EtOH, 0.2 volumes 10 M NH4Ac, recovered by centrifugation, washed in 70% EtOH, dried (SpeedVac), and resuspended in 30 μl of Mung bean nuclease buffer (30 mM NaAc, pH 4.6, 300 mM NaCl, 1 mM ZnSO4, 0.35 mM DTT, 2% glycerol) containing 25 units of Mung bean nuclease (Pharmacia). The single-stranded hair-pin DNA was clipped by incubating the reaction at 30° C. for 30 min, followed by addition of 70 μl 10 mM Tris-Cl, pH 7.5, 1 mM EDTA, phenol extraction, and ethanol precipitation with 2 volumes of 96% EtOH and 0.1 volumes 3M NaAc, pH 5.2 on ice for 30 min.

The ds cDNAs were recovered by centrifugation (20000 rpm, 30 min.), and blunt-ended with T4 DNA polymerase in 30 μl of T4 DNA polymerase buffer (20 mM Tris-acetate, pH 7.9, 10 mM MgAc, 50 mM KAc, 1 mM DTT) containing 0.5 mM each dNTP and 5 units of T4 DNA polymerase (New England Biolabs) by incubating the reaction mixture at +16° C. for 1 hour. The reaction was stopped by addition of EDTA to 20 mM final concentration, followed by phenol and chloroform extractions and ethanol precipitation for 12 hours at −20° C. by adding 2 volumes of 96% EtOH and 0.1 volumes of 3M NaAc, pH 5.2.

After the fill-in reaction the cDNAs were recovered by centrifugation as above, washed in 70% EtOH, and the DNA pellet was dried in SpeedVac. The cDNA pellet was resuspended in 25 μl of ligation buffer (30 mM Tris-Cl, pH 7.8, 10 mM MgCl2, 10 mM DTT, 0.5 mM ATP) containing 2 μg EcoRI adaptors (0.2 μg/μl, Pharmacia) and 20 units of T4 ligase (Promega) by incubating the reaction mix at +16° C. for 12 hours. The reaction was stopped by heating at +65° C. for 20 min, and then on ice for 5 min. The adapted cDNA was digested with Not I restriction enzyme by addition of 20 μl autoclaved water, 5 μl of 10× Not I restriction enzyme buffer (New England Biolabs) and 50 units of NotI (New England Biolabs), followed by incubation for 3 hours at +37° C. The reaction was stopped by heating the sample at +65° C. for 15 min. The cDNAs were size-fractionated by agarose gel electrophoresis on a 0.8% SeaPlaque GTG low melting temperature agarose gel (FMC) in 1×TBE (in autoclaved water) to separate unligated adaptors and small cDNAs. The gel was run for 12 hours at 15 V, the cDNA was size-selected with a cut-off at 0.7 kb by cutting out the lower part of the agarose gel. Then a 1.5% agarose gel was poured in front of the cDNA-containing gel, and the ds cDNAs were concentrated by running the gel backwards until it appeared as a compressed band on the gel. The cDNA-containing gel piece was cut out from the gel and the cDNA was extracted from the gel using the GFX gel band purification kit (Amersham-Pharmacia Biotech) as follows. The trimmed gel slice was weighed in a 2 ml Biopure Eppendorf tube, then 10 ml of Capture Buffer was added for each 10 mg of gel slice, the gel slice was dissolved by incubation at 60° C. for 10 min, until the agarose was completely solubilized, the sample at the bottom of the tube by brief centrifugation. The melted sample was transferred to the GFX spin column placed in a collection tube, incubated at 25° C. for 1 min., and then spun at full speed in a microcentrifuge for 30 seconds. The flow-trough was discarded, and the column was washed with 500 μl of wash buffer, followed by centrifugation at full speed for 30 seconds.

The collection tube was discarded, and the column was placed in a 1.5 ml Eppendorf tube, followed by elution of the cDNA by addition of 50 μl TE, pH 7.5 to the center of the column, incubation at 25° C. for 1 min., and finally by centrifugation for 1 min at maximum speed. The eluted cDNA was stored at −20° C. until library construction.

Preparation of EcoRI/NotI-Cleaved Vector for Library Construction

A plasmid DNA preparation for an EcoRI-NotI insert-containing pYES 2.0 cDNA clone, was purified using a Qiagen Tip-100 according to the manufacturer's instructions. 10 μg of purified plasmid DNA was digested to completion with NotI and EcoRI in a total volume of 60 μl by addition of 6 μl of 10× NEBuffer for EcoRI (New England Biolabs), 40 units of Not I (New England Biolabs), and 20 units of EcoRI (New England Biolabs) followed by incubation for 6 hours at +37° C. The reaction was stopped by heating the sample at +65° C. for 20 min. The digested plasmid DNA was extracted once with phenol-chloroform, then with chloroform, followed by ethanol precipitation for 12 hours at −20° C. by adding 2 volumes of 96% EtOH and 0.1 volumes of 3M NaAc, pH 5.2. The precipitated DNA was resuspended in 25 μl 1×TE, pH 7.5, loaded on a 0.8% SeaKem agarose gel in 1×TBE (in autoclaved H2O), and run on the gel for 3 hours at 60 V. The digested vector was cut out from the gel, and the DNA was extracted from the gel using the GFX gel band purification kit (Amersham-Pharmacia Biotech) according to the manufacturers instructions. After measuring the DNA concentration by OD260/280, the eluted vector was stored at −20° C. until library construction.

Construction of the Aspergillus oryzae IFO4177 cDNA Library

To establish the optimal ligation conditions for the cDNA library, four test ligations were carried out in 10 μl of ligation buffer (30 mM Tris-Cl, pH 7.8, 10 mM MgCl₂, 10 mM DTT, 0.5 mM ATP) containing 7 μl ds cDNA, (corresponding to approximately 1/10 of the total volume in the cDNA sample), 2 units of T4 ligase (Promega) and 25 ng, 50 ng and 75 ng of EcoRI-NotI cleaved pYES 2.0 vector, respectively (Invitrogen). The vector background control ligation reaction contained 75 ng of EcoRI-NotI cleaved pYES 2.0 vector without cDNA. The ligation reactions were performed by incubation at +16° C. for 12 hours, heated at 65° C. for 20 min, and then 10 μl of autoclaved water was added to each tube. One μl of the ligation mixtures was electroporated (200 W, 2.5 kV, 25 mF) to 40 ill electrocompetent E. coli DH10B cells (Bethesda Research Laboratories). After addition of 1 ml SOC to each transformation mix, the cells were grown at +37° C. for 1 hour, 50 μl and 5 μl from each electroporation were plated on LB+ampicillin plates (100 mg/ml) and grown at +37° C. for 12 hours. Using the optimal conditions, an A. oryzae A1560 cDNA library containing 2.5×107 independent colony forming units was established in E. coli, with a vector background of ca.1%. The cDNA library was stored as individual pools (25000 c.f.u./pool) in 20% glycerol at −80° C.

Plasmid DNA from individual colonies was isolated using the Qiaprep system and was sequenced on an ABI 3700 Capillary sequencer according to the manufacturer's instructions. A comparison of the sequences to the SWISSPROT database a cDNA cloned, named pJaL621, was identified to code for a cellobiase. Sequencing of the 2771 bp cDNA clone (SEQ ID NO:1) revealed an open reading frame encoding a polypeptide of 861 amino acids (SEQ ID NO:2) with a calculated molecular mass of 93.437 Da.

### Example 2

Expression of the Aspergillus oryzae Cellobiase in Aspergillus oryzae

Construction of an A. oryzae Cellobiase Expression Plasmid

The Aspergillus expression plasmid pCaHj527 (disclosed in WO 00/70064) consists of an expression cassette based on the Aspergillus niger neutral amylase II promoter fused to the Aspergillus nidulans triose phosphate isomerase non translated leader sequence (Pna2/tpi) and the Aspergillus niger amyloglycosidase terminater (Tamg). Also present on the plasmid is the Aspergillus selective marker amdS from Aspergillus nidulans enabling growth on acetamide as sole nitrogen source and the URA3 marker from Saccharomyces cerevisiae enabling growth of the pyrF defective Escherichia coli strain DB6507 (ATCC 35673). Transformation into E. coli DB6507 using the S. cerevisiae URA 3 gene as selective marker was done in the following way:

E. coli DB6507 was made competent by the method of Mandel and Higa (Mandel, M. and A. Higa (1970) J. Mol. Biol. 45, 154). Transformants were selected on solid M9 medium (Sambrook et. al (1989) Molecular cloning, a laboratory manual, 2. edition, Cold Spring Harbor Laboratory Press) supplemented with 1 g/l casaminoacids, 500 μg/l thiamine and 10 mg/l kanamycin.

pCaHj527 was modified in the following way:
The Pna2/tpi promoter present on pCaHj527 was subjected to site directed mutagenises by a simple PCR approach;
Nucleotide 134-144 was altered from SEQ ID NO:3 to SEQ ID NO:4 using the mutagenic primer 141223 (SEQ ID NO: 5);
Nucleotide 423-436 was altered from SEQ ID NO:6 to SEQ ID NO:7 using the mutagenic primer 141222 (SEQ ID NO: 8).
   The resulting plasmid was termed pMT2188.

The cellobiase gene was cloned into pMT2188 in the following way:

The coding region of the A. oryzae cellobiase was amplified by PCR, using the following two oligonucleotide primers: B2902E12 (SEQ ID NO:9) and B2902F01 (SEQ ID NO:10). To facilitate cloning a restriction enzyme site was inserted into the 5′ end of each primer; primer B2902E12 contains a BglII site and primer B2902F01 contains an XhoI site.

The A. oryzae cDNA clone pJaL621 was used as template in the PCR reaction. The reaction was performed in a volume of 100 μl containing 2.5 units Taq polymerase, 100 ng of pJaL621, 250 nM of each dNTP, and 10 pmol of each of the two primers described above in a reaction buffer of 50 mM KCl, 10 mM Tris-HCl pH 8.0, 1.5 mM MgCl2.

Amplification was carried out in a Perkin-Elmer Cetus DNA Termal 480, and consisted of one cycle of 3 minutes at 94° C., followed by 25 cycles of 1 minute at 94° C., 30 seconds at 55° C., and 1 minute at 72° C. The PCR reaction produced a single DNA fragment of 2615 bp in length. This fragment was digested with BglII and XhoI and isolated by gel electrophoresis, purified, and cloned into pMT2188 digested with BamHI and XhoI, resulting in a plasmid, which was designated pJaL660. Thus, the construction of the plasmid pJaL660 resulted in a fungal expression plasmid for the A. oryzae cellobiase cDNA gene.

Expression of the A. oryzae Cellobiase in Aspergillus oryzae

The strains BECh2 (disclosed in WO 00/30322) was transformed with pJaL660 as described by Christensen et al.; Biotechnology 1988, 6, 1419-1422. Typically, A. oryzae mycelia were grown in a rich nutrient broth. The mycelia were separated from the broth by filtration. The enzyme preparation Novozyme® (Novozymes A/S) was added to the mycelia in osmotically stabilizing buffer such as 1.2 M MgSO4 buffered to pH 5.0 with sodium phosphate. The suspension was incubated for 60 minutes at 37° C. with agitation. The protoplast was filtered through mira-cloth to remove mycelial debris. The protoplast was harvested and washed twice with STC (1.2 M sorbitol, 10 mM CaCl₂, 10 mM Tris-HCl pH 7.5). The protoplasts were finally resuspended in 200-1000 μl STC.

For transformation 5 μg DNA was added to 100 μl protoplast suspension and then 200 μl PEG solution (60% PEG 4000, 10 mM CaCl₂, 10 mM Tris-HCl pH 7.5) was added and the mixture is incubated for 20 minutes at room temperature. The protoplast were harvested and washed twice with 1.2 M sorbitol. The protoplast was finally resuspended 200 μl 1.2 M sorbitol, plated on selective plates (minimal medium+10 g/l Bacto-Agar (Difco), and incubated at 37° C. After 3-4 days of growth at 37° C., stable transformants appear as vigorously growing and sporulating colonies. Transformants was spore isolated twice.

Transformants were grown in shake flask for 4 days at 30° C. in 100 ml YPM medium (2 g/l yeast extract, 2 g/l peptone, and 2% maltose). Supernatants (20 μl) were analyzed on SDS page gel (Novex NuPAGE 10% Bis-Tris gel) according to the manufacturers instructions. The transformant no. 4 was named JaL406.

### Example 3

Production of the A. oryzae Cellobiase

The transformed Aspergillus oryzae JAL406 was grown in a fermentor using standard substrate and after 5 days of incubation the fermentation broth was harvested. The whole broth was passed through a Glass fiber filter from Whatman, first a “D” filter then a “F” filter and finally the clear enzyme solution was passed through a sterile filter from Millipore with a pore size of 22 μm. The mycelium was discarded. The clear enzyme solution was concentrated using a Filtron cross flow membrane with a cut off of 10 kDa. For obtaining a pure enzyme the concentrate was passed over a 2 l Sephacryl 200 column equilibrated in 0.1 M Na-Acetate pH 6 buffer. The pure enzyme has a molecular weight of 91 kDa in SDS-PAGE. The melting temperature in DSC at pH 6.0 was 67° C. For comparison the purified A. niger cellobiase has a melting temperature of 62° C. The molar extinction coefficient of the A. oryzae cellobiase was 169540.

The A. niger cellobiase has a 3 fold lower catalytic activity compared to the A. oryzae cellobiase at pH 4.0 and 40° C. when using paranitrophenyl beta-D-glucose as substrate. Both the A. oryzae and A niger cellobiases showed more than 50% catalytic activity between pH 3.0 and 7.0.

The cloned A. oryzae cellobiase was produced in a much higher yield after being transformed back into A. oryzae using the strong promotor and terminator genes.

Deposit of Biological Material

The following biological material has been deposited under the terms of the Budapest Treaty with the DSMZ-Deutsche Sammlung von Mikroorganismen und Zellkulturen GmbH, Mascheroder Weg 1B, D-38124 Braunschweig, Germany, and given the following accession number:

| Deposit | Accession Number | Date of Deposit |
|---|---|---|
| NN049573 | DSM 14240 | 2001-04-19 |

[image]

1-31. (canceled)

32. A polypeptide having cellobiase activity, selected from the group consisting of:
(a) a polypeptide comprising an amino acid sequence which has at least 80% identity with amino acids 1 to 842 of SEQ ID NO:2 or which has at least 90% identity with amino acids 1 to 351 of SEQ ID NO:2;
(b) a polypeptide comprising an amino acid sequence which has at least 80% identity with the polypeptide encoded by the cellobiase encoding part of the nucleotide sequence inserted into a plasmid present in E. coli DSM 14240; and
(c) a polypeptide which is encoded by a nucleotide sequence which hybridizes under medium stringency conditions with a polynucleotide probe selected from the group consisting of
(i) the complementary strand of nucleotides 87 to 2612 of SEQ ID NO:1, and
(ii) the complementary strand of nucleotides 87 to 1139 of SEQ ID NO:1.

33. The polypeptide according to claim 32, comprising an amino acid sequence which has at least 80% identity with amino acids 1 to 842 of SEQ ID NO:2.

34. The polypeptide according to claim 32, comprising an amino acid sequence which has at least 85% identity with amino acids 1 to 842 of SEQ ID NO:2.

35. The polypeptide according to claim 32, comprising an amino acid sequence which has at least 90% identity with amino acids 1 to 842 of SEQ ID NO:2.

36. The polypeptide according to claim 32, comprising an amino acid sequence which has at least 95% identity with amino acids 1 to 842 of SEQ ID NO:2.

37. The polypeptide according to claim 32, comprising an amino acid sequence which has at least 99% identity with amino acids 1 to 842 of SEQ ID NO:2.

38. The polypeptide according to claim 32, which comprises the amino acids 1 to 842 of SEQ ID NO:2.

39. The polypeptide according to claim 32, which consists of the amino acids 1 to 842 of SEQ ID NO:2.

40. The polypeptide according to claim 32, comprising an amino acid sequence which has at least 80% identity with the polypeptide encoded by the cellobiase encoding part of the nucleotide sequence inserted into a plasmid present in E. coli DSM 14240.

41. The polypeptide according to claim 32, comprising an amino acid sequence which has at least 85% identity with the polypeptide encoded by the cellobiase encoding part of the nucleotide sequence inserted into a plasmid present in E. coli DSM 14240.

42. The polypeptide according to claim 32, comprising an amino acid sequence which has at least 90% identity with the polypeptide encoded by the cellobiase encoding part of the nucleotide sequence inserted into a plasmid present in E. coli DSM 14240.

43. The polypeptide according to claim 32, comprising an amino acid sequence which has at least 95% identity with the polypeptide encoded by the cellobiase encoding part of the nucleotide sequence inserted into a plasmid present in E. coli DSM 14240.

44. The polypeptide according to claim 32, comprising an amino acid sequence which has at least 99% identity with the polypeptide encoded by the cellobiase encoding part of the nucleotide sequence inserted into a plasmid present in E. coli DSM 14240.

45. The polypeptide according to claim 32, which comprises the amino acid sequence encoded by the cellobiase encoding part of the nucleotide sequence inserted into a plasmid present in E. coli DSM 14240.

46. The polypeptide according to claim 32, which consists of the amino acid sequence encoded by the cellobiase encoding part of the nucleotide sequence inserted into a plasmid present in E. coli DSM 14240.

47. The polypeptide according to claim 32, which is encoded by a nucleotide sequence which hybridizes under medium stringency conditions with a polynucleotide probe selected from the group consisting of:
(i) the complementary strand of nucleotides 87 to 2612 of SEQ ID NO:1, and
(ii) the complementary strand of nucleotides 87 to 1139 of SEQ ID NO:1.

48. The polypeptide according to claim 32, which is encoded by a nucleotide sequence which hybridizes under high stringency conditions with a polynucleotide probe selected from the group consisting of:
(i) the complementary strand of nucleotides 87 to 2612 of SEQ ID NO:1, and
(ii) the complementary strand of nucleotides 87 to 1139 of SEQ ID NO:1.

49. A nucleotide sequence which encodes for the polypeptide defined claim 32.

50. A nucleic acid construct comprising the nucleotide sequence defined in claim 49 operably linked to one or more control sequences that direct the production of the polypeptide in a suitable host.

51. A recombinant expression vector comprising the nucleic acid construct defined in claim 50.

52. A recombinant host cell comprising the nucleic acid construct defined in claim 50.

53. A method for producing a polypeptide as defined in claim 32, the method comprising:
(a) cultivating a strain, which in its wild-type form is capable of producing the polypeptide, to produce the polypeptide; and
(b) recovering the polypeptide.

54. A method for producing a polypeptide as defined in any of claims 32, the method comprising:
(a) cultivating a recombinant host cell as defined in claim 52 under conditions conducive for production of the polypeptide; and
(b) recovering the polypeptide.


SEQUENCE LISTING (10 sequences)

SEQ ID NO: 1 - DNA, 2771 bp, Aspergillus oryzae
  CDS (30)..(2612)
  mat_peptide (87)..()
ctgttctgctggttacctgccacgttatcatgaagcttggttggatcgaggtggccgcattggcggctgcctcagtagtcagtgccaaggatgatctcgcgtactcccctcctttctacccttccccatgggcagatggtcagggtgaatgggcggaagtatacaaacgcgctgtagacatagtttcccagatgacgttgacagagaaagtcaacttaacgactggaacaggatggcaactagagaggtgtgttggacaaactggcagtgttcccagactcaacatccccagcttgtgtttgcaggatagtcctcttggtattcgtttctcggactacaattcagctttccctgcgggtgttaatgtcgctgccacctgggacaagacgctcgcctaccttcgtggtcaggcaatgggtgaggagttcagtgataagggtattgacgttcagctgggtcctgctgctggccctctcggtgctcatccggatggcggtagaaactgggaaggtttctcaccagatccagccctcaccggtgtactttttgcggagacgattaagggtattcaagatgctggtgtcattgcgacagctaagcattatatcatgaacgaacaagagcatttccgccaacaacccgaggctgcgggttacggattcaacgtaagcgacagtttgagttccaacgttgatgacaagactatgcatgaattgtacctctggcccttcgcggatgcagtacgcgctggagtcggtgctgtcatgtgctcttacaaccaaatcaacaacagctacggttgcgagaatagcgaaactctgaacaagcttttgaaggcggagcttggtttccaaggcttcgtcatgagtgattggaccgctcatcacagcggcgtaggcgctgctttagcaggtctggatatgtcgatgcccggtgatgttaccttcgatagtggtacgtctttctggggtgcaaacttgacggtcggtgtccttaacggtacaatcccccaatggcgtgttgatgacatggctgtccgtatcatggccgcttattacaaggttggccgcgacaccaaatacacccctcccaacttcagctcgtggaccagggacgaatatggtttcgcgcataaccatgtttcggaaggtgcttacgagagggtcaacgaattcgtggacgtgcaacgcgatcatgccgacctaatccgtcgcatcggcgcgcagagcactgttctgctgaagaacaagggtgccttgcccttgagccgcaaggaaaagctggtcgcccttctgggagaggatgcgggttccaactcgtggggcgctaacggctgtgatgaccgtggttgcgataacggtacccttgccatggcctggggtagcggtactgcgaatttcccatacctcgtgacaccagagcaggcgattcagaacgaagttcttcagggccgtggtaatgtcttcgccgtgaccgacagttgggcgctcgacaagatcgctgcggctgcccgccaggccagcgtatctctcgtgttcgtcaactccgactcaggagaaagctatcttagtgtggatggaaatgagggcgatcgtaacaacatcactctgtggaagaacggcgacaatgtggtcaagaccgcagcgaataactgtaacaacaccgtggtcatcatccactccgtcggaccagttttgatcgatgaatggtatgaccaccccaatgtcactggtattctctgggctggtctgccaggccaggagtctggtaactccatcgccgatgtgctgtacggtcgtgtcaaccctggcgccaagtctcctttcacttggggcaagacccgggagtcgtatggttctcccttggtcaaggatgccaacaatggcaacggagcgccccagtctgatttcacccagggtgttttcatcgattaccgccatttcgataagttcaatgagacccctatctacgagtttggctacggcttgagctacaccaccttcgagctctccgacctccatgttcagcccctgaacgcgtcccgatacactcccaccagtggcatgactgaagctgcaaagaactttggtgaaattggcgatgcgtcggagtacgtgtatccggaggggctggaaaggatccatgagtttatctatccctggatcaactctaccgacctgaaggcatcgtctgacgattctaactacggctgggaagactccaagtatattcccgaaggcgccacggatgggtctgcccagccccgtttgcccgctagtggtggtgccggaggaaaccccggtctgtacgaggatcttttccgcgtctctgtgaaggtcaagaacacgggcaatgtcgccggtgatgaagttcctcagctgtacgtttccctaggcggcccgaatgagcccaaggtggtactgcgcaagtttgagcgtattcacttggccccttcgcaggaggccgtgtggacaacgacccttacccgtcgtgaccttgcaaactgggacgtttcggctcaggactggaccgtcactccttaccccaagacgatctacgttggaaactcctcacggaaactgccgctccaggcctcgctgcctaaggcccagtaaggggcaagtcctgattgtacagagcatttcgagatttatgatgtacatgtttatgaatgacctagggtagggtaatacttagtagggttagttctaattcttggagtcaagtattgactcactgggccgataaaaaaaaaaaaaaaaaaaaaaaaa

SEQ ID NO: 2 - protein, 861 aa, Aspergillus oryzae
MKLGWIEVAALAAASVVSAKDDLAYSPPFYPSPWADGQGEWAEVYKRAVDIVSQMTLTEKVNLTTGTGWQLERCVGQTGSVPRLNIPSLCLQDSPLGIRFSDYNSAFPAGVNVAATWDKTLAYLRGQAMGEEFSDKGIDVQLGPAAGPLGAHPDGGRNWEGFSPDPALTGVLFAETIKGIQDAGVIATAKHYIMNEQEHFRQQPEAAGYGFNVSDSLSSNVDDKTMHELYLWPFADAVRAGVGAVMCSYNQINNSYGCENSETLNKLLKAELGFQGFVMSDWTAHHSGVGAALAGLDMSMPGDVTFDSGTSFWGANLTVGVLNGTIPQWRVDDMAVRIMAAYYKVGRDTKYTPPNFSSWTRDEYGFAHNHVSEGAYERVNEFVDVQRDHADLIRRIGAQSTVLLKNKGALPLSRKEKLVALLGEDAGSNSWGANGCDDRGCDNGTLAMAWGSGTANFPYLVTPEQAIQNEVLQGRGNVFAVTDSWALDKIAAAARQASVSLVFVNSDSGESYLSVDGNEGDRNNITLWKNGDNVVKTAANNCNNTVVIIHSVGPVLIDEWYDHPNVTGILWAGLPGQESGNSIADVLYGRVNPGAKSPFTWGKTRESYGSPLVKDANNGNGAPQSDFTQGVFIDYRHFDKFNETPIYEFGYGLSYTTFELSDLHVQPLNASRYTPTSGMTEAAKNFGEIGDASEYVYPEGLERIHEFIYPWINSTDLKASSDDSNYGWEDSKYIPEGATDGSAQPRLPASGGAGGNPGLYEDLFRVSVKVKNTGNVAGDEVPQLYVSLGGPNEPKVVLRKFERIHLAPSQEAVWTTTLTRRDLANWDVSAQDWTVTPYPKTIYVGNSSRKLPLQASLPKAQ

SEQ ID NO: 3 - DNA, 11 bp, synthetic construct
gtactaaaacc

SEQ ID NO: 4 - DNA, 11 bp, synthetic construct
ccgttaaattt

SEQ ID NO: 5 - DNA, 45 bp, synthetic construct
ggatgctgttgactccggaaatttaacggtttggtcttgcatccc

SEQ ID NO: 6 - DNA, 14 bp, synthetic construct
atgcaatttaaact

SEQ ID NO: 7 - DNA, 14 bp, synthetic construct
cggcaatttaacgg

SEQ ID NO: 8 - DNA, 44 bp, synthetic construct
ggtattgtcctgcagacggcaatttaacggcttctgcgaatcgc

SEQ ID NO: 9 - DNA, 27 bp, synthetic construct
agatctaccatgaagcttggttggatc

SEQ ID NO: 10 - DNA, 24 bp, synthetic construct
ctcgagttactgggccttaggcag